(19) United States
(12) Patent Application Publication
(10) Pub. No.: US 2026/0061166 A1
(43) Pub. Date: Mar. 5, 2026

(54) DISENGAGEABLE CATHETER ASSEMBLIES

(71) Applicant: YEDA RESEARCH AND DEVELOPMENT CO. LTD., Rehovot (IL)

(72) Inventors: Brian BERKOWITZ, Rehovot (IL); Yaniv SHILO, Rehovot (IL)

(21) Appl. No.: 19/367,036

(22) Filed: Oct. 23, 2025

Related U.S. Application Data
(63) Continuation of application No. PCT/IL2024/050433, filed on May 7, 2024.

(30) Foreign Application Priority Data
May 10, 2023 (IL) 302841

(51) Int. Cl. A61M 25/04; A61M 25/00

(52) U.S. Cl. CPC A61M 25/04; A61M 25/0023; A61M 2202/0014; A61M 2202/0496; A61M 2210/1082

(57) ABSTRACT

The present disclosure relates to catheter assemblies designed to provide fluid communication between an organ in a patient's body and the external environment. Such a catheter assembly can include an indwelling catheter equipped with an anchoring mechanism at a distal portion thereof, configured to transition between a compacted configuration and an expanded configuration, and an external tube configured to frictionally engage with a proximal end portion of the indwelling catheter. The frictional force between corresponding engagement surfaces of the indwelling catheter and the external tube is configured to allow disengagement therebetween in response to a pull force equal to or greater than a threshold separating pull force, applied to the external tube, wherein the threshold separating pull force can be designed to be lower than the pull force required to cause dislodgment of the indwelling catheter from the patient's body.

### CROSS-REFERENCE TO RELATED APPLICATIONS

This application is a continuation of International Application No. PCT/IL2024/050433, filed May 7, 2024, which claims the benefit of Israel Application No. 302841, filed May 10, 2023, which is incorporated by reference herein.

### FIELD

The present disclosure relates to catheter assemblies that provide fluid communication between a patient's body cavity and an external receptacle or reservoir, such as nephrostomy catheters, feeding catheters or peritoneal dialysis catheters, and more precisely, to catheter assemblies that include an indwelling catheter frictionally engaged with and disengageable from an external tube.

### BACKGROUND

Various catheters and catheter assemblies are widely used to provide fluid communication between a cavity in a patient's body and an external receptacle or reservoir. One example of a common medical application for such catheters are nephrostomy tubes, placed directly into the kidney through the patient's flank to drain urine. Indications for nephrostomy include both short and long term medical conditions. For example, nephrostomy may be placed for several weeks due to ureteral obstruction by an impacted stone or obstructing stone with signs of infections. More chronic conditions such as ureteral strictures and obstructing tumors (usually in morbid patients) require nephrostomy placement for months to years.

Such catheters usually include an anchoring mechanism, placed within the target cavity in a patient's body to anchor the tube in position for the duration of implantation. One type of an anchoring mechanism includes a curled pigtail configuration at the portion of the tube that resides in the kidney, while the remainder of the tube has a relatively linear length that exists in the abdomen and is connected to a urine bag. In some implementations, the nephrostomy tube is inserted over a guidewire in to the kidney, and as the guidewire is removed, the end of the tube curls into the pigtail shape. In other implementations, a tensioning member such as a wire or a string is passed along the tube and attached to a distal end thereof, wherein pulling on one end of the wire or string serves to curve the distal portion of the tube to the pigtail shape. Additional types of anchoring mechanisms use Foley type inflatable balloons or Malecot-type designs with outwardly bendable ribs or wings rather than a pigtail to anchor the tube in the kidney. Urine drains from the kidney and is collected in the bag, which is then periodically emptied or replaced.

The main drawback of most indwelling catheter, and in particular, of nephrostomy catheters, is accidental dislodgement. For example, while the pigtail is intended to act as a stabilizing anchor within the kidney, its flexibility also means that, with sudden or steady tugging, the pigtail can be straightened and the nephrostomy tube removed. In other cases, the pigtail may be forcibly pulled while maintaining its curved shape, causing severe damage to the kidney and flank of the patient while being forced through the organs and tissues. Similarly, a radially expanded Malecot tube or a Foley type nephrostomy tube, which includes a relatively small-sized balloon due to the limited volume available in the kidney's internal cavity, can also be dislodged. As a consequence, nephrostomy tubes are frequently pulled out, either accidentally (e.g., leaving the collection bag on the counter while brushing teeth, then just walking away), or intentionally (e.g., patients suffering from dementia). Regardless of the reason, premature or unintended removal of a nephrostomy tube is a serious event, and without replacement, urine accumulation in the kidney with inadequate drainage can lead to urine leakage through the flank, hydronephrosis, and renal failure.

Hospital emergency room visits following premature or inadvertent removal of a nephrostomy tube are numerous; on a weekly basis, a large medical center may deal with cases on a daily basis. These cases necessitate hospital emergency department visits; treatment generally requires anesthesia and insertion of a new tube. As such, the demand on medical staff time and resources is significant, given the frequency of such occurrences and required treatment.

### SUMMARY

The present disclosure is directed toward catheter assemblies that include an indwelling tube and an external tube frictionally engageable therewith, wherein the external tube is disengageable from the indwelling catheter upon application of a pulling force which is lower than a force required to dislodge the indwelling catheter from a patient's body.

According to some aspects of the disclosure, there is provided a disengageable catheter assembly comprising an indwelling catheter and an external tube. The indwelling catheter comprises a catheter wall defining a catheter lumen, wherein the indwelling catheter extends between a catheter distal portion and a catheter proximal expanded portion. The catheter distal portion comprises an anchoring mechanism configured to transition between a compacted configuration and an expanded configuration, wherein the catheter distal portion comprises at least one catheter distal opening in fluid communication with the catheter lumen. The catheter proximal expanded portion comprises a catheter varying-diameter engagement surface. The external tube comprises a tube wall defining a tube lumen, wherein the external tube comprises a tube distal expanded portion which comprises a tube varying-diameter engagement surface.

The disengageable catheter assembly is configured to non-adjustably transition between an engaged state, in which the catheter engagement surface and the tube engagement surface are frictionally engaged with each other, and a disengaged state, in which the indwelling catheter and the external tube are separated from each other. The catheter lumen is in fluid communication with the tube lumen in the engaged state. The frictional engagement between the catheter varying-diameter engagement surface and the tube varying-diameter engagement surface in the engaged state is configured to allow disengagement therebetween when the indwelling catheter and the external tube are subjected to an axial pull force exceeding a threshold separating force that is not greater than 20 N for a duration of no more than 10 seconds. The disengageable catheter assembly is manually reconnectable from the disengaged state to the engaged state.

The various innovations of this disclosure can be used in combination or separately. This summary is provided to introduce a selection of concepts in a simplified form that are further described below in the detailed description. This summary is not intended to identify key features or essential features of the claimed subject matter, nor is it intended to be used to limit the scope of the claimed subject matter. The foregoing and other objects, features, and advantages of the invention will become more apparent from the following detailed description, which proceeds with reference to the accompanying figures.

### BRIEF DESCRIPTION OF THE FIGURES

Some examples of the invention are described herein with reference to the accompanying figures. The description, together with the figures, makes apparent to a person having ordinary skill in the art how some examples may be practiced. The figures are for the purpose of illustrative description and no attempt is made to show structural details of an example in more detail than is necessary for a fundamental understanding of the invention. For the sake of clarity, some objects depicted in the figures are not to scale.

In the Figures:

FIG. 1 shows an exemplary indwelling catheter connected to a patient's body.

FIG. 2A shows an exemplary disengageable catheter assembly in a disengaged state.

FIG. 2B shows the disengageable catheter assembly of FIG. 2A in an engaged state.

FIGS. 3A-3C are cross-sectional views of a portion of an exemplary disengageable catheter assembly in different states of engagement, having a catheter proximal expanded portion shaped as a prolate spheroid, and a tube distal expanded portion shaped as a prolate hemispheroid.

FIG. 4 shows an exemplary disengageable catheter assembly having a catheter proximal expanded portion shaped as a prolate hemispheroid and a tube distal expanded portion shaped as a prolate spheroid.

FIG. 5 shows a portion of an exemplary catheter assembly with a rough catheter varying-diameter engagement surface.

FIG. 6 shows a portion of an exemplary catheter assembly with a rough tube varying-diameter engagement surface.

FIG. 7 shows a portion of an exemplary catheter assembly with a friction layer.

FIG. 8 shows a portion of an exemplary catheter assembly with multi-segmented catheter proximal expanded portion and tube distal expanded portion, having a plurality of curved segments.

FIGS. 9A and 9B show a portion of an exemplary catheter assembly having frustoconical segments, in a disengaged and an engaged state, respectively.

FIG. 10 shows a portion of an exemplary catheter assembly with multi-segmented catheter proximal expanded portion and tube distal expanded portion, having a plurality of frustoconical segments.

FIGS. 11A and 11B show a portion of an exemplary catheter assembly with circumferential protrusions, in a disengaged and an engaged state, respectively.

FIGS. 12A and 12B show a portion of an exemplary catheter assembly with axial protrusions, in a disengaged and an engaged state, respectively.

FIGS. 13A-13D show different stages of utilizing an exemplary catheter assembly with a pigtail catheter.

FIGS. 14A-14B shows steps in utilization of a levered locker of an exemplary catheter assembly.

FIGS. 15A-15B show steps in utilization of a frictionally movable ring of an exemplary catheter assembly.

FIGS. 16A-16C show steps in utilization of a spool of an exemplary catheter assembly.

FIGS. 17A-17B show stages of utilizing an exemplary catheter assembly with a pigtail catheter having a catheter proximal side opening through which a flexible tensioning member extends.

FIGS. 18A and 18B show a portion of an exemplary catheter assembly with ball-and-socket type of engagement, in a disengaged and an engaged state, respectively.

FIG. 19 shows a partial view of a distal section of a Foley catheter.

FIG. 20 shows a partial view of a distal section of a Malecot catheter.

### DETAILED DESCRIPTION

For purposes of this description, certain aspects, advantages, and novel features of the examples of this disclosure are described herein. The disclosed methods, apparatus, and systems should not be construed as being limiting in any way. Instead, the present disclosure is directed toward all novel and nonobvious features and aspects of the various disclosed examples, alone and in various combinations and sub-combinations with one another. The methods, apparatus, and systems are not limited to any specific aspect or feature or combination thereof, nor do the disclosed examples require that any one or more specific advantages be present, or problems be solved. The technologies from any example can be combined with the technologies described in any one or more of the other examples. In view of the many possible examples to which the principles of the disclosed technology may be applied, it should be recognized that the illustrated examples are only preferred examples and should not be taken as limiting the scope of the disclosed technology.

Although the operations of some of the disclosed examples are described in a particular, sequential order for convenient presentation, it should be understood that this manner of description encompasses rearrangement, unless a particular ordering is required by specific language set forth below. For example, operations described sequentially may in some cases be rearranged or performed concurrently. Moreover, for the sake of simplicity, the attached figures may not show the various ways in which the disclosed methods can be used in conjunction with other methods. Additionally, the description sometimes uses terms like “provide” or “achieve” to describe the disclosed methods. These terms are high-level abstractions of the actual operations that are performed. The actual operations that correspond to these terms may vary depending on the particular implementation and are readily discernible by one of ordinary skill in the art.

All features described herein are independent of one another and, except where structurally impossible, can be used in combination with any other feature described herein.

As used in this application and in the claims, the singular forms “a,” “an,” and “the” include the plural forms unless the context clearly dictates otherwise. Additionally, the terms “have” or “includes” means “comprises”. Further, the terms “engaged”, “connected”, “coupled”, and “attached”, as used herein, are interchangeable and generally mean physically and/or mechanically coupled or linked and does not exclude the presence of intermediate elements between the coupled or associated items absent specific contrary language. As used herein, “and/or”means “and”or “or”, as well as “and”and “or”.

Directions and other relative references may be used to facilitate discussion of the drawings and principles herein, but are not intended to be limiting. For example, certain terms may be used such as “inner,” “outer,” “upper,” “lower,” “inside,” “outside,”, “top,” “bottom,” “interior,” “exterior,” “left,” right,“ and the like. Such terms are used, where applicable, to provide some clarity of description when dealing with relative relationships, particularly with respect to the illustrated examples. Such terms are not, however, intended to imply absolute relationships, positions, and/or orientations. For example, with respect to an object, an “upper” part can become a “lower” part simply by turning the object over. Nevertheless, it is still the same part and the object remains the same.

The term “plurality” or “plural” when used together with an element means two or more of the element. Directions and other relative references (e.g., inner and outer, upper and lower, above and below, left and right, and proximal and distal) may be used to facilitate discussion of the drawings and principles herein but are not intended to be limiting.

It should be understood that the disclosed examples can be adapted to direct fluids from an external source into a cavity of a patient's body, and/or to drain fluid from a cavity of a patient's body to an external bag or other receptacle.

Throughout the figures of the drawings, different superscripts for the same reference numerals are used to denote different examples of the same elements. Examples of the disclosed devices and systems may include any combination of different examples of the same elements. Specifically, any reference to an element without a superscript may refer to any alternative example of the same element denoted with a superscript. In order to avoid undue clutter from having too many reference numbers and lead lines on a particular drawing, some components will be introduced via one or more drawings and not explicitly identified in every subsequent drawing that contains that component.

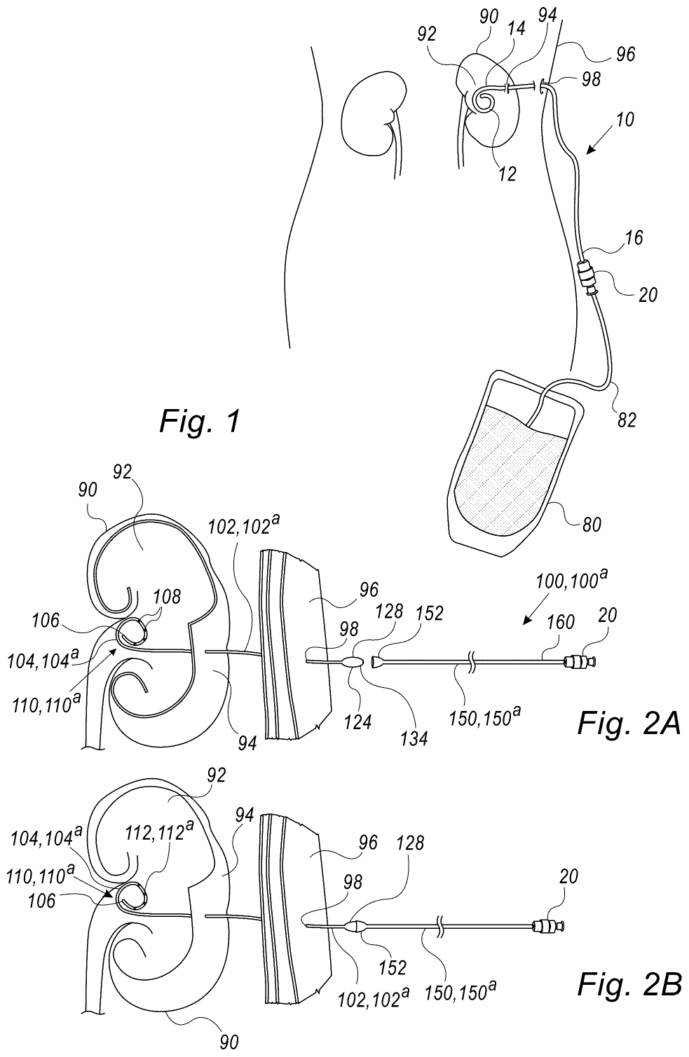

FIG. 1 illustrates an exemplary conventional indwelling catheter 10 connected to a patient's body and configured to provide fluid communication between an organ 90 within a patient's body and an extracorporeal receptacle, such as a drain bag 80. The exemplary indwelling catheter 10 shown in FIG. 1 can be a nephrostomy catheter, also known as a nephrostomy tube, configured to drain urine from the kidney 90 into a drain bag 80. A nephrostomy catheter 10 is a type of a drainage catheter that can be percutaneously inserted through a body point of entry 98 along skin surface 96, through the organ wall 94, which is the kidney wall 94 in the illustrated example, and into the organ cavity 92, which is the fluid collection space inside the kidney.

Since indwelling catheters 10 are frequently intended to remain positioned within the cavity 92 of a patient's body, such as, but not limited to, a cavity of an organ 90 (e.g., kidney, liver, stomach, etc.), for extended time periods (e.g., days, weeks or months), such catheters usually include a catheter distal end portion 14 equipped with an anchoring mechanism 12 configured to retain the catheter distal end portion 14 within the patient's body, and more specifically, within a body cavity 92. In the example illustrated in FIG. 1, the anchoring mechanism 12 is implemented as a curled end portion, also known as a pigtail formation, being confined to within the body cavity 92. The indwelling catheter 10 includes an interior or indwelling portion, residing in the patient body between the body point of entry 98 and the catheter distal end portion 14, and an exterior portion disposed out of the patient's body, between the body point of entry 98 and a catheter proximal end portion 16.

A conventional indwelling catheter 10 can further include a connector 20 at the catheter proximal end portion 16, utilized to connect it to additional accessories, such as to a drain bag tube 82 leading into a drain bag 80. Such connector 20, which can be implemented as Luer connectors or other types of connectors, usually require manual operation for both connecting and disconnecting the catheter to the external tubes or other accessories, and are mostly designed to prevent spontaneous or unintentional disengagement unless manually manipulated for that purpose. Thus, when excessive pull force is applied to the indwelling catheter 10, for example by a pull force applied to the drain bag 80 or any other accessory connected to catheter 10, the connector will conventionally retain string engagement between the catheter 10 and any accessory connected thereto, such that the pull force is transferred to the catheter distal end portion 14 and the anchoring mechanism 12, acting against the organ wall 94. If this force is high enough, the indwelling catheter 10 may be dislodged from the organ 90, which can damage the organ 90 and/or other organs in the patient's body, interrupt with proper fluid communication with the body cavity 92, and pose a risk to the patient which may necessitate complicated interventional procedures.

While an exemplary nephrostomy catheter is illustrated in FIG. 1, dislodgement is not limited to nephrostomy catheters. Virtually any medical tube or catheter which is mounted to a patient for an extended period of time to provide fluid communication between a body cavity 92 and an external source or target can be dislodged either passively due to unintentional snagging or tangling of the catheter or tube, or by confused, obtunded or otherwise uncooperative patients.

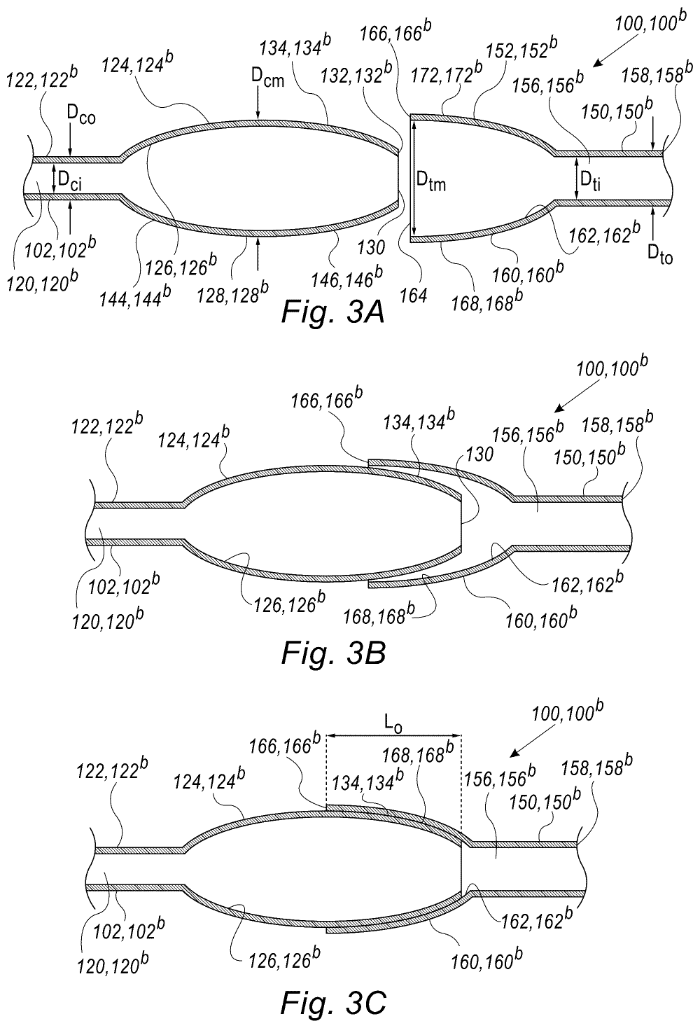

FIGS. 2A and 2B show an exemplary disengageable catheter assembly 100, illustrated in a disengaged state in FIG. 2A, and in an engaged state in FIG. 2B. Disengeagable catheter assembly 100 includes an indwelling catheter 102, extending between a catheter distal portion 104 and a catheter proximal expanded portion 128, and an external tube 150 extending between a tube distal expanded portion 152 and a tube proximal end portion 154. Indwelling catheter 102 comprises a catheter wall 122 defining a catheter lumen 120 (indicated, for example, in FIGS. 3A-3C), and the external tube 150 comprises a tube wall 158 defining a tube lumen 156 (indicated, for example, in FIGS. 3A-3C). The catheter proximal expanded portion 128 is frictionally engageable with the tube distal expanded portion 152, such that in the engaged state, shown in FIG. 2A for example, the catheter lumen 120 is in fluid communication with the tube lumen 156.

As used herein, the term “indwelling” when characterizing a catheter generally refers to a catheter that is at least partially disposed within a portion of a patient's body. In some cases, indwelling catheters may be temporarily implanted or inserted for a time period of months, as may be for implantation or insertion of urinary or nephrostomy catheters. In some cases, implantation or insertion is for a time period between a minute and months. Generally, the time period of implantation or insertion is dependent upon the indwelling catheter being implanted or inserted and the needs of the subject.

The terms “catheter” and “tube”, as used herein, refer to a thin, flexible tube or cannula that can be extruded from medical grade materials, including an outer wall which is flexible at least in a portion if not over most if not all of its length, and a lumen extending from one end to another end of the catheter or tube, such as between a proximal and a distal end thereof. The term “indwelling catheter” refers to a catheter or tube intended for insertion into a patient's body, such that the catheter distal portion resides within a body cavity, while the catheter proximal end portion resides outside the patient's body. In contrast, the term “external tube” refers to a tube intended to reside completely out of the patient's body, such that both of the tube distal and portion and the tube proximal end portion are disposed outside the body.

The terms “indwelling catheter 102” and “catheter 102”, as used throughout the specification, with or without superscripts, are interchangeable. The terms “external tube 150” and “tube 150”, as used throughout the specification, with or without superscripts, are interchangeable. The terms “disengageable catheter assembly 100” and “catheter assembly 100”, as used throughout the specification, with or without superscripts, are interchangeable.

As used herein, the terms “proximal” and “distal” refer to the direction closer to and away from, respectively, a practitioner who would implant or insert the indwelling catheter into a patient's body. Thus, for example, the end of the indwelling catheter 102 first touching the skin surface 96 of the patient would be the distal end, while the opposite end of the catheter (e.g., the end of the catheter being manipulated by the practitioner) would be the proximal end. When the external tube 150 is connected to the indwelling catheter 102, together forming a continuous disengageable catheter assembly 100 as shown for example in FIG. 2B, the distal end of the indwelling catheter is also the distal end of the disengageable catheter assembly, and the proximal end of the external tube may be also referred to as the proximal end of the disengageable catheter assembly. The term “proximal” when used with two spatially separated positions or parts of an object can be understood to mean closer to or oriented towards the proximal end of the disengageable catheter assembly. The term “distal” when used with two spatially separated positions or parts of an object can be understood to mean closer to or oriented towards the distal end of the disengageable catheter assembly. The terms “longitudinal” and “axial” are interchangeable, and refer to an axis extending in the proximal and distal directions, unless otherwise expressly defined.

The term “organ”, as used herein, refers to an internal organ in a patient's body into which or from which materials may be transferred, via catheter and/or tube lumens for example, from/to an external location. Exemplary organs may include, but are not limited to: bladder, kidney, heart, intestines, uterus, lungs, urinary tract, and the like. The term “body cavity”, as used herein, refers to an inner cavity in a patient's body, which can be a cavity of an organ, or another cavity such as the peritoneal cavity, the abdominal cavity, and the like.

The indwelling catheter 102 comprises at least one catheter distal opening 108. In some examples, the at least one catheter distal opening 108 includes an opening at the distal tip 106 of the catheter. In some examples, the at least one catheter distal opening 108 includes one or more side openings extending through the thickness of the catheter wall 122 at the catheter distal portion 104. For simplicity, any reference throughout the specification to “catheter distal openings 108” will refer to either a single catheter distal opening 108 or to a plurality of catheter distal openings 108, unless stated otherwise.

The catheter distal openings 108 are exposed to the surrounding environment, such as the body cavity 92, and are in fluid communication with catheter lumen 120, thus providing fluid communication between body cavity 92 and catheter lumen 120. The indwelling catheter 102 terminates at a catheter proximal end opening 130, opposite to catheter distal tip 106, such that catheter lumen 120 extends from catheter distal tip 106 to catheter proximal end opening 130.

The indwelling catheter 102 further comprises an anchoring mechanism 110, for example at the catheter distal portion 104, configured to retain catheter distal portion 104 within body cavity 92. Various types of anchoring mechanism 110 can be implemented, such as in the form of a pigtail, inflatable balloons, Malecot ribs, and the like, some of which will be described in greater detail hereinbelow. An anchoring mechanism 110 is usually configured to assume a wider profile when positioned within body cavity 92, for example when compared to the diameter of the indwelling catheter 102, such as at the portion extending through the organ wall 94. The wider profile of the anchoring mechanism 110 can press against the inner surface of organ wall 94, preventing it from slipping out of the organ 90. An indwelling catheter 102 will include an actuating mechanism configured to transition the anchoring mechanism 110 between a compacted configuration and the expanded configuration. The anchoring mechanism will usually be kept in the compacted configuration during delivery toward the organ and the implantation procedure, and the actuation mechanism will be utilized to expand the anchoring mechanism 110 once it is situated within the body cavity 92.

The external tube 150 comprises tube distal end opening 164 (indicated, for example, in FIG. 3A), from which the tube lumen 156 proximally extends toward the tube proximal end portion 154. The tube proximal end portion 154 can be open ended, or include additional components such as valves, connectors 20, and the like. In the example illustrated in FIG. 2A, external tube 150 is shown to include a connector 20 by which it can be connected to additional external accessories, such as a drain bag 80 and/or drain bag tube 82 leading to a drain bag. However, it is to be understood that a connector 20 is shown by way of illustration and not limitation, and that in some implementations, the external tube 150 can serve as a drain bag tube that includes a drain bag 80 at its proximal end portion. In some examples, external tube 150 can be connectable, such as via connector 20, to a syringe or a pump.

The disengageable catheter assembly 100 is configured to transition between an engaged state, in which external tube 150 is connected to indwelling catheter 102 (see FIG. 2B) by a friction force, and a disengaged state, in which external tube 150 is disconnected from indwelling catheter 102 (see FIG. 2A). Specifically, the tube distal expanded portion 152 is configured to connect with the catheter proximal expanded portion 128 by a friction force, in a binary or non-adjustable manner, as will be described in further detail below. When frictionally connected to each other in the engaged state, fluid communication is formed between catheter lumen 120 and tube lumen 156, such that fluid can be either drained therethrough, entering through catheter distal openings 108 into catheter lumen 120, and flowing continuously therefrom and through tube lumen 156, optionally to a drain bag 80 or other external receptacle, when the disengageable catheter assembly 100 is implemented as a drain tube (e.g., nephrostomy tube or urine tube). Similarly, fluid can be fed from an external source into tube lumen 156 and through catheter lumen 120, to exit into body cavity 92 via one or more catheter distal opening(s) 108, for example when the disengageable catheter assembly 100 is implemented as a supply tube (e.g., feed tube), and/or when cleaning fluid is injected (for example, periodically) to clean or wash the lumens. The frictional connection can be further configured to create a fluid tight seal between external tube 150 and indwelling catheter 102 in the engaged state, so as to prevent any fluid, including any liquid or gas, from escaping.

The catheter proximal expanded portion 128 comprises a catheter varying-diameter engagement surface 134, and the tube distal expanded portion 152 comprises a tube varying-diameter engagement surface 168. When the disengageable catheter assembly 100 is in the disengaged state, the distal tube expanded portion 150 can be slid over and/or into the catheter proximal expanded portion 128, such that tube varying-diameter engagement surface 168 overlaps with and contacts the catheter varying-diameter engagement surface 134, forming a interference engagement, such as in the form of a frictional force, holding the tube 150 and catheter 102 connected in the engaged state, as long as the disengageable catheter assembly 100 is not subjected to a pulling force exceeding a disengagement threshold value, as described hereinbelow.

In some implementations, catheter varying-diameter engagement surface 134 is a portion of a surface of catheter wall 122 along catheter proximal expanded portion 128, and tube varying-diameter engagement surface 168 is a portion of a surface of tube wall 158 along tube distal expanded portion 152, such that the external tube 150 and the indwelling catheter 102 are configured to frictionally connect with each other without the need for any other intermediate connectors therebetween. Specifically, some types of prior-art catheter assemblies can include two tubes and/or catheter connectable to each other via dedicated connectors, which can be separate components or components attached to an end of one of the tubes/catheters. Most prior-art connectors, such as connector 20 schematically illustrated between indwelling catheter 10 and drain bag tube 82 in FIG. 1, are designed to connect both tubes/catheters in a manner that prevents passive disengagement from each other. For example, connectors that include Luer connectors, threaded connection mechanisms, snap-fit mechanisms, latched locks, and the like, are usually designed to retain a locked connection, and are disengageable from each other only by intentional or deliberate manual manipulation, such as a user or operator unlocking the connection, for example by unscrewing them from each other when threadedly engaged, releasing a latch when locked thereby, and the like. In some implementations, the disengageable catheter assembly 100 is devoid of connectors designed to facilitate connection of external tube 150 and indwelling catheter 102 to each other in any type of engagement which is not frictional engagement.

While most conventional connections between tubes and/or catheters are designed to resist separation when subjected to an axial force lower than a minimum threshold value, engagement between catheter 102 and tube 150 of assemblies 100 disclosed herein is configured to allow separation between the two when subjected to relatively lower axial forces. For example, the standard ISO 80369-7:2016 Clause 6.4 specifies that small-bore Luer slip connectors shall not separate from each other when subjected to an axial force between 23 N (Newton) and 25 N for a period between 10 and 15 seconds. This can be performed by attaching the male and female portions of the tested connector to an automated tensile strength tester, such as those provided by the company Instron®, with the above-mentioned test parameters. In contrast to such test parameters, the frictional engagement between catheter proximal expanded portion 128 and tube distal expanded portion 152 of any exemplary assembly 100 disclosed herein, is configured to allow separation therebetween when subjected to an axial load at a lower threshold of 20 N or less, for a period of no more than 10 seconds, and in some examples, for a period of no more than 5 seconds, utilizing a similar test setup as described in standard ISO 80369-7:2016 and standard ISO 80369-20:2016 for testing axial force separation of small-bore Luer slip connectors.

It is to be understood that the term “frictional engagement”, as used herein, refers to friction fit or press fit engagement between catheter 102 and tube 150, wherein friction or press fit engagements include any of: engagement between one component 102 or 150 having an inner diameter equal to or slightly less than an outer diameter of another component 150 or 102, such as at any point of contact between an engagement surface 134 of the catheter 102 and an engagement surface 168 of the tube 150; engagement of at least one rough surface with another surface; engagement between a surface of a friction layer of one component 102 or 150 with a surface of another component 150 or 102; engagement between protrusions of one component 102 or 150 with a surface of another component 150 or 102; and any combination thereof.

When the disengageable catheter assembly 100 is in the engaged state, the external tube 150 and the indwelling catheter 102 are configured to disconnect from each other when a pull force exceeds a threshold separating force. In other words, the friction force between both, and more specifically, between catheter varying-diameter engagement surface 134 and tube varying-diameter engagement surface 168, is selected to allow disengagement therebetween only in response to a pulling force equal to or greater than a threshold separating force. The threshold value of the separating pull force is selected to be high enough to prevent spontaneous disengagement between catheter 102 and tube 150, but is less than the pulling force required to dislodge the indwelling catheter 102 from the patient's organ 90 and/or body.

For example, accidental snagging or catching the external tube 150 or another component coupled thereto (e.g., a separate urine bag or urine bag tube) applies an axial pulling force of a magnitude that, if applied directly to the indwelling catheter, would cause dislodgment thereof from the organ 90. However, the frictional engagement between the tube 150 and catheter 102 is designed to yield at a pulling force which is lower than the force that would cause such dislodgement, meaning that when a pulling force equal to or exceeding a predetermined threshold separating force, which is lower than the dislodgment-facilitating force, the tube 150 slides away from and is separated from the indwelling catheter 102, leaving the indwelling catheter 102 in position, mitigating risk of dislodgement.

Some prior-art catheter assemblies include frangible or breakable connectors configured to break in response to a pulling force exceeding a predetermined threshold value. A potential drawback of such solutions is that once such a connector breaks, a more complicated procedure is required to replace the connector to reattach the tubes/catheters to each other. In contrast, once external tube 150 is decoupled from indwelling catheter 102, for example in response to a pulling force equal to or exceeding the threshold separating force, it can be easily reconnected by manually sliding it back over indwelling catheter 102, transitioning the disengageable catheter assembly 100 back to an engaged state in a relatively simplified, immediate and uncomplicated manner. In some implementations, the disengageable catheter assembly 100 is devoid of a frangible connector configured to break in response to a predetermined threshold pulling force.

A disengageable catheter assembly 100 of any example described throughout the current specification and the claims is manually reconnectable. The term “manually reconnectable”, as used throughout the specification and the claims, refers to the external tube 150 and the indwelling catheter 102 being manually connectable to each other by the user, such as by hand, when the catheter proximal expanded portion 128 and the tube distal expanded portion 152 are slid into each other, either prior to first use or after the catheter 102 and tube 150 were disconnected from each other (for example, after being subjected to a pull force exceeding the threshold separating force), without the need to use specialized tools or equipment.

The proposed disengagement mechanism is also unique compared to other prior-art frictional engagement interfaces between tubes, which can be with or without intermediate connectors, in that that conventional frictional engagement between tubes/catheters is usually designed to retain engagement even at relatively high pulling forces, such that dislodgment of an indwelling catheter will still occur when the friction force is high enough to resist separation at dislodgment pull forces. In contrast, the disengageable catheter assembly 100 disclosed herein allows separation of external tube 150 from indwelling catheter 102 when external tube 150 is subjected to an axially oriented pulling force lower than the force required for dislodgement, wherein the separation is advantageously reversible, allowing the tube 150 to be easily manually reconnected by the catheter 102 still residing in the patient, without requiring professional intervention from medical personnel.

As mentioned, connection of external tube 150 to indwelling catheter 102 can be easily and conveniently performed manually by any user, including the patient, by distally sliding or pushing the tube distal expanded portion 152 until its tube varying-diameter engagement surface 168 sufficiently overlaps and/or contacts and/or engages the catheter varying-diameter engagement surface 134, so as to achieve the desired frictional force therebetween. In some implementations, a cap (not shown) can be further provided to seal the catheter proximal end opening 130 in the disengaged state. For example, when external tube 150 is pulled away from indwelling catheter 102, the indwelling catheter 102 can be capped to prevent leak through catheter proximal end opening 130 in this state. The cap can be removed, in such cases, prior to connecting or re-connecting external tube 150 to the indwelling catheter 102. In some examples, a cap for sealing the catheter proximal end opening 130 has a shape complementary to that of the catheter proximal expanded portion 128. For example, such a cap can have a shape that is generally similar to the shape of the corresponding tube distal expanded portion 152, configured to be placed over the catheter proximal expanded portion 128, wherein the cap is enclosed at its central proximal end.

As mentioned above, dislodgement is not limited to nephrostomy catheters, and while examples of disengageable catheter assembly 100 are described throughout the current disclosure with reference to nephrostomy catheters, it is to be understood that a disengageable catheter assembly 100 disclosed herein can be implemented as any other type of catheter assembly mounted to a patient to provide fluid communication between a body cavity 92 and the external environment. The target body cavity 92, which can be, in some cases, a cavity of a target organ 90, is thus also selected according to the type of catheter assembly 100 is implemented as. In some examples, the disengageable catheter assembly 100 is implemented as a nephrostomy catheter configured to drain urine from the kidney 90. In some examples, disengageable catheter assembly 100 is implemented as a urine catheter configured to drain urine from the bladder 90. In some examples, disengageable catheter assembly 100 is implemented as a feeding tube, such as a gastric feeding tube configured to provide nutrition into the stomach 90, as well as nasojejunal or jejunostomy feeding tubes configured to provide nutrition into the jejunum 90 or the duodenum. In some examples, disengageable catheter assembly 100 is utilized to administer medicine into the patient body. In some examples, disengageable catheter assembly 100 is utilized to supply oxygen to a patient in need thereof. In some examples, the disengageable catheter assembly 100 is implemented as a peritoneal dialysis catheter configured to direct fluid (e.g., cleansing fluid) into and out of the peritoneal cavity 92.

Engagement between indwelling catheter 102 and external tube 150 according to any example described herein is non-adjustable or binary, meaning that frictional engagement is achieved when the entire catheter varying-diameter engagement surface 134 is in direct contact with the entire corresponding tube varying-diameter engagement surface 168, while partial contact between part of any of these engagement surfaces 134, 168 with each other is not sufficient to keep the catheter 102 and tube 150 coupled to each other. This also means that the threshold separation force can be selected to bring part of the engagement surfaces 134, 168 away from each other, which effects full separation of the tube 150 from the catheter 102 since the complete surface area is required to maintain both in an engaged state.

Thus, any reference to engagement of an assembly 100 being non-adjustable or binary, means that engagement force between catheter 102 and 150 is not meant to be adjusted by being able to slide one into the other to a different length that can increase or decrease the total surface of engagement contact between the two, but is rather limited to a single engaged state by which the entire areas of engagement surfaces 134, 168 are in contact with each other in a manner that is sufficient to keep them coupled to each other, without enabling this surface areas to be increased while in the engaged state, and wherein a decrease in the contact areas leads to disengagement between the two. Non-adjustable engagement can allow for increased safety of use, particularly when used by a patient or other non-clinical personnel, without the need to consider the amount of surface area contact required to properly coupled the components to each other to maintain them properly engaged. Nevertheless, it is to be understood that even when referred to as binary of non-adjustable engagement, in the specification and the claims, a limited amount of deviation from a specific contact area required for engagement maintenance may be possible to account for manufacturing tolerance, such as in the range of ±10% of the contact area defined by any of the engagement surfaces 134, 168.

The threshold separating force can be selected to be lower than any one of several undesired failure mechanisms, such as, but not limited to: (1) the force required to deform or damage an anchoring mechanism 110 of the indwelling catheter 102, such that the anchoring mechanism 110 no longer provides adequate anchoring within the organ 90; (2) the force required to pull the anchoring mechanism 110 away from the organ 90 even if not completely deformed, posing a risk of tearing through the organ wall 94; (3) for indwelling catheters which are further sutured to the patient's skin around the body point of entry 98, the force required to tear such sutures; and (4) any combination of (1), (2) and/or (3), including referring to one of the values, such as the lower of the values.

As mentioned, the threshold separating force can be further influenced by characteristics of the patient, which can include, but are not limited to: BMI (Body Mass Index), health, skin tone and degree of fat/tissue layers—particularly in the region of body point of entry 98. For low BMI and favorable skin tone, higher forces may be required to cause dislodgment or facilitate other modes of failure as described above. Repeated catheterization through the same or close body point of entry 98, as well as poor skin or tissue tone, can cause detachment (or suture/tissue tearing) at relatively low forces.

In some examples, the threshold separating force is not higher than 5 N. In some examples, the threshold separating force is not higher than 10 N, such as being set to a value of 7 N or 8 N. In some examples, the external tube 150 is configured to separate from the indwelling catheter 102 in response to a separating pull force of at least 5 N. In some examples, the external tube 150 is configured to separate from the indwelling catheter 102 in response to a separating pull force of at least 8 N.

In some examples, the threshold separating force is not higher than 20 N, such as being set to a value of 15 N, 17 N or 20 N. In some examples, the external tube 150 is configured to separate from the indwelling catheter 102 in response to a separating pull force of at least 15 N. In some examples, the external tube 150 is configured to separate from the indwelling catheter 102 in response to a separating pull force of at least 17 N. In some examples, the external tube 150 is configured to separate from the indwelling catheter 102 in response to a separating pull force of about 20 N. If a force that is higher than the preset threshold, such as a force that is greater than 20 N, is required for separation of the external tube 150 from the indwelling catheter 102, this may lead to an undesired risk of the indwelling catheter 102 being dislodged from the patient's body when subjected to such forces, prior to separation of the tube 150, thereby putting the patient at risk which is similar to that of conventional nephrostomy catheters.

It is to be understood that any reference to the external tube 150 configured to separate from catheter 102 at a separating pull force exceeding a specified value, both in the specification and the claims, refers to the external tube 150 configured to necessarily separate from catheter 102 as soon as the pulling force reaches or slightly exceeds the specified value. For example, if the external tube 150 is configured to separate from catheter 102 when subjected to a pull force of 10 N, this means that it will necessarily separate from catheter 102 at any pull force that is equal to or greater than 10 N.

Testing procedures for separation between catheter 102 and tube 150 can be performed by attaching free ends of the indwelling catheter 102 and the external tube 150 to an automated tensile strength tester, such as those provided by the company Instron®, with the catheter proximal expanded portion 128 and the tube distal expanded portion 152 frictionally engaged with each other, and subjecting the assembly 100 to an axial pull force following test requirements similar to those described in standard ISO 80369-7:2016 and standard ISO 80369-20:2016 for testing axial force separation of small-bore Luer slip connectors, wherein the test parameters require the tube 150 and catheter 102 to separate from each other when an axial load equal to the separating pull force is applied thereto for a duration of no more than 10 seconds, and in some examples, for a duration of no more than 5 seconds.

While tube 150 is configured to necessarily separate from catheter 102 when subjected to the separating pull force, it may be also desired to retain engagement at pull forces which are lower than the force required to facilitate separation. Thus, the external tube 150 can be further configured to retain frictional engagement with the indwelling catheter 102 when subjected to any pull force that does not exceed the maximal non-separating pull force. The maximal non-separating pull force will be lower than the separating pull force.

In some examples, the maximal non-separating pull force is about 95% of the separating pull force. In some examples, the maximal non-separating pull force is equal to about 90% of the minimal separating pull force. In some examples, the maximal non-separating pull force is equal to about 85% of the minimal separating pull force. In some examples, the maximal non-separating pull force is equal to about 80% of the minimal separating pull force. In some examples, the maximal non-separating pull force is equal to about 75% of the minimal separating pull force.

The term “about”, as used herein, means plus or minus 10% of the value stated. For example, about 90% of a separating pull force of 10 N would include 8.1 N and 9.9 N. Nevertheless, a maximal non-separating pull force cannot be equal to or exceed the value of a separating pull force. Thus, a maximal non-separating pull force which is about 95% of a separating pull force of 10 N will include 8.55 N and values below 10 N, but will not include any value equal to or greater than 10 N.

When the indwelling catheter 102 is inserted into a patient's body, the catheter distal portion 104 resides inside of the patient's body, such as inside the target organ cavity 92 from which fluids are to be drained, while the catheter proximal expanded portion 128 and the entirety of the external tube 150 are configured to remain outside the patient's body. In some cases, it may be desired to keep a relatively short portion of the indwelling catheter 102 extending proximally outward from the patient's body (i.e., from the body point of entry 98), to minimize chances of this portion being snagged, caught, or otherwise pulled, in a manner that may apply axially pull forces directly to the indwelling catheter 102. In some examples, the length of the indwelling catheter 102 is dimensioned such that the portion extending from the body point of entry 98 to the catheter proximal end opening 130, when implanted in a patient, does not exceed a maximal exterior catheter length. The maximal catheter exterior length, which includes the catheter proximal expanded portion 128 residing outside the patient's body, can be selected to allow the catheter proximal expanded portion 128 to be conveniently manually gripped and stabilized by the patient or other user when connecting the tube distal expanded portion 152 thereto. In some examples, the maximal exterior catheter length is about 15 cm (centimeters). In some examples, the maximal exterior catheter length is about 10 cm. In some examples, the maximal exterior catheter length is about 5 cm.

Various exemplary implementations for disengageable catheter assemblies 100 and/or components thereof can be referred to, throughout the specification, with superscripts, for ease of explanation of features that refer to such exemplary implementations. It is to be understood, however, that any reference to structural or functional features of any assembly or component, without a superscript, refer to these features being commonly shared by all specific exemplary implementations that can be also indicated by superscripts. In contrast, features emphasized with respect to an exemplary implementation of any assembly or component, including catheter assemblies 100, referred to with a superscript, may be optionally shared by some but not necessarily all other exemplary implementations. For example, disengageable catheter assembly 100^{a}, which is indicated in FIGS. 2A-2B as well, is an exemplary implementation of disengageable catheter assembly 100, and thus includes all of the features described for disengageable catheter assembly 100 throughout the current disclosure, except that while the indwelling catheter 102 of disengageable catheter assembly 100 can include different types of anchoring mechanisms 110, disengageable catheter assembly 100^{a} includes an indwelling catheter which is implemented as a pigtail catheter 102^{a}, provided with an anchoring mechanism 110^{a} that comprises a pigtail 112.

The term “pigtail”, as used herein, refers to a loop structure formed in a catheter distal portion 104. The loop structure can be a partial loop, a complete loop, or more than one loop (i.e., defining one or more coils of the pigtail).

A plurality of catheter distal openings 108 can be formed along the pigtail 112, optionally spaced from each other as shown. Inclusion of a plurality of distal openings 108 can advantageously increase the intake area within the body cavity 92.

As mentioned above, an actuation mechanism is provided to transition any type of anchoring mechanism 110 from a compacted configuration, in which it assumes a relatively narrow profile for easier delivery through the patient's body during the implantation procedures, to the expanded configuration once positioned in the target body cavity 92. In the case of a pigtail catheter 102^{a}, at least two different actuation mechanisms are contemplated, as will be explained hereinbelow.

In some implementations, the catheter distal portion 104^{a} is shape-set to assume a natural pigtail-shaped curved configuration, also referred to as an expanded configuration of the anchoring mechanism 110^{a} that includes a pigtail 112. In practice, a user (e.g., clinician) may temporarily reduce the profile of catheter distal portion 104^{a}, by using a suitable device such as a stylet, guidewire, guidewire catheter, and the like, to temporarily straighten the catheter distal portion 104^{a}. In this compacted configuration, the indwelling catheter 102^{a} can be introduced into the patient's body, and advanced until catheter distal portion 104^{a} reside within the cavity 92 of the target organ 90. The straightening device (e.g., stylet, guidewire, and the like) may then be retrieved from catheter lumen 120, allowing catheter distal portion 104^{a} to revert to its curved or coiled pigtail-shaped configuration, which increases the profile of anchoring mechanism 110^{a}, preventing it from slipping out of the body cavity 92. The tendency of the catheter distal portion 104^{a} to assume a natural curved configuration may be achieved, for example, by bending catheter distal portion 104^{a} into a desired curved shape during the manufacturing process, before the indwelling catheter 102^{a} is fully cured or cooled, or by incorporating shape memory materials (e.g., Nitinol) into catheter distal portion 104^{a}.

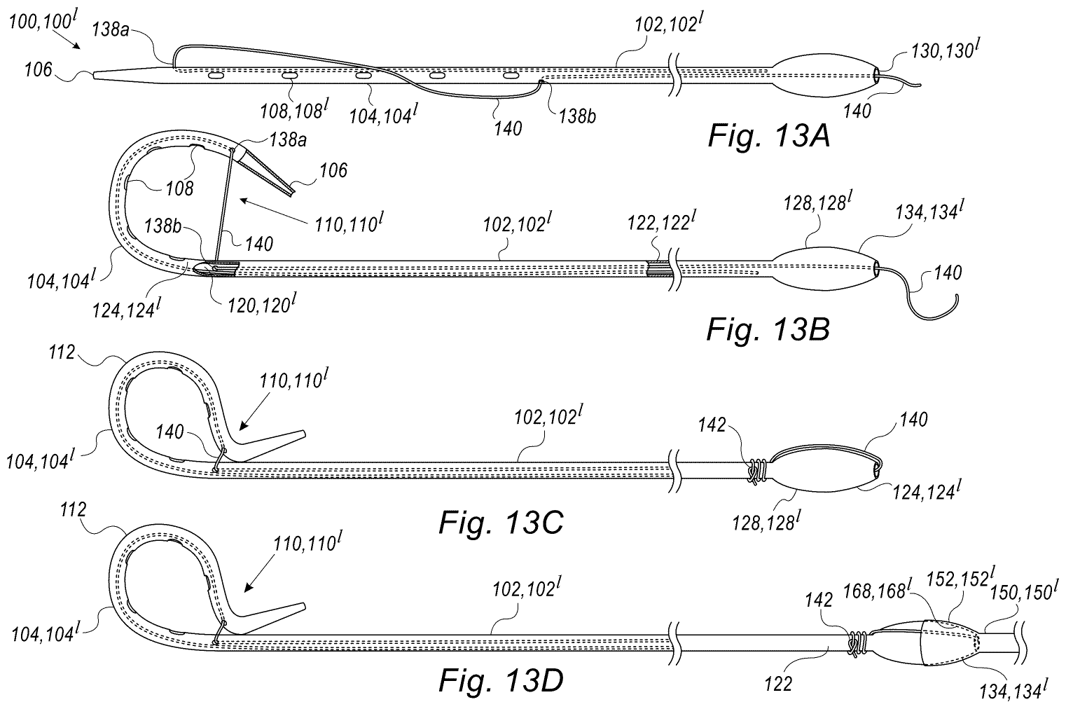

In some implementations, an anchoring mechanism 110 that includes a pigtail 112 is configured to assume a curved configuration not necessarily by shape-setting catheter distal portion 104 but rather by an actuating mechanism that includes a flexible tensioning member, such as a wire, suture, or string, attached to the catheter distal portion 104 and pullable from catheter proximal end portion 128, as will be elaborated in greater detail hereinbelow with respect to FIGS. 13A-13D.

FIGS. 3A-3C are cross-sectional views of a portion of an exemplary disengageable catheter assembly 100^{b} in different states of engagement. Catheter assembly 100^{b} is an exemplary implementation of catheter assembly 100, and thus includes all of the features described for catheter assembly 100 and any components thereof throughout the current disclosure, except that while the external tube 150 and indwelling catheter 102 of catheter assembly 100 can have various shapes of expanded portion 128 and 152, the catheter proximal expanded portion 128^{b} of indwelling catheter 102^{b} has a prolate spheroid shape (i.e., barrel-shaped), and the tube distal expanded portion 152^{b} has a prolate hemispheroid shape (i.e., cup-shaped), configured to slide over the catheter proximal expanded portion 128^{b} and facilitate frictional engagement therebetween.

The catheter wall 122 of indwelling catheter 102 defines inner and outer surfaces of the catheter 102. For example, the catheter wall 122 defines, at the region of the catheter proximal expanded portion 128, a catheter expanded portion inner surface 126 facing catheter lumen 120 inside of proximal expanded portion 128, and an opposite catheter expanded portion outer surface 124 facing away from catheter lumen 120. The catheter has a tubular portion extending distally from the catheter proximal end portion 128, wherein the tubular portion of the catheter 102 defines a catheter outer diameter D_{co} and a catheter inner diameter D_{ci}. In some examples, the indwelling catheter 102 has a uniform diameter along the tubular portion, in which case the catheter outer diameter D_{co} and catheter inner diameter D_{ci} are uniform outer and inner diameters along the tubular portion of the indwelling catheter 102. It is to be understood that catheter 102 can include, in some examples, a tapering distal tip 106, which does not exclude the tubular portion of catheter 102 from being termed to have uniform diameters, but rather that in such cases, the tubular portion of the catheter 102 has uniform outer and inner diameters D_{co} and D_{ci}, respectively, along the a length that extends between the optionally tapering catheter distal tip 106 and the catheter proximal expanded portion 128.

The tube wall 158 of external tube 150 defines inner and outer surfaces of the external tube 150. For example, the tube wall 122 defines, at the region of tube distal expanded portion 152, a tube expanded portion inner surface 162 facing tube lumen 156 inside of distal expanded portion 152, and an opposite tube expanded portion outer surface 160 facing away from tube lumen 156. The external tube has a tubular portion extending proximally from the tube distal expanded portion 152, wherein the tubular portion of the external tube 150 defines a tube outer diameter Dₜₒ and a tube inner diameter Dₜᵢ. In some examples, the external tube 150 has a uniform diameter along the tubular portion, in which case the tube outer diameter Dₜₒ and tube inner diameter Dₜᵢ are uniform outer and inner diameters along the tubular portion of the external tube 150.

The catheter wall 122 terminates at catheter proximal edge 132 which surrounds catheter proximal end opening 130. The tube wall 158 terminates at tube distal edge 166 which surrounds the tube distal end opening 164. Catheter engagement surface 134 can extend, in some examples, along at least a portion of the catheter proximal end portion 128. The catheter engagement surface 134 can extend, in some examples, from the catheter proximal edge 132 in a distal direction, along at least a portion of the catheter proximal end portion 128. Tube engagement surface 168 can extend along at least a portion of the tube distal expanded portion 152. The tube engagement surface 168 can extend, in some examples, from the tube distal edge 166 in a proximal direction, along at least a portion of the tube distal expanded portion 152. In some examples, the length and/or surface area of the catheter engagement surface 134 is substantially equal to the length and/or surface area, respectively, of the tube engagement surface 168.

The term “substantially equal”, with reference to dimension parameters such as lengths, means that both parameters can be within a range of ±10% from each other. For example, when the length of the catheter engagement surface 134 is termed to be substantially equal to the length of the tube engagement surface 168, it means that the length of the catheter engagement surface 134 can have a value between 90% and 110% of the length of the tube engagement surface 168, inclusive.

In some examples, as illustrated for catheter assembly 100^{b}, the catheter proximal expanded portion 128 is shaped to include a catheter outwardly expanding segment 144 extending proximally from the tubular portion of catheter 102, and a catheter inwardly tapering segment 146 extending from the catheter outwardly expanding segment 144 and terminating at catheter proximal edge 132. The outer diameter of the catheter outwardly expanding segment 144 can gradually increase in a proximal direction from the catheter outer diameter D_{co} of the catheter's tubular portion, to a maximal catheter engagement surface diameter D_{cm}. The outer diameter of the catheter inwardly tapering segment 146 can gradually decrease in a proximal direction from the maximal catheter engagement surface diameter D_{cm} to a smaller diameter at the catheter proximal edge 132.

In some examples, as illustrated for catheter assembly 100^{b}, the tube distal expanded portion 152 is shaped to include a tube outwardly expanding segment 172 extending distally from the tubular portion of the external tube 150, optionally terminating at the tube distal edge 166. The inner diameter of the tube outwardly expanding segment 172 can gradually increase in a distal direction from the tube inner diameter Dₜᵢ of the external tube's 150 tubular portion, to a maximal tube engagement surface diameter Dₜₘ, which can be optionally defined at the tube distal edge 166.

FIG. 3A shows the catheter assembly 100^{b} in a disengaged state, with the catheter proximal expanded portion 128^{b} and the tube distal expanded portion 152^{b} shown to be axially spaced from each other. FIG. 3B shows an intermediate state during engagement, with the tube distal expanded portion 152^{b} pushed towards and over the catheter proximal expanded portion 128^{b}. In this state, even though a portion of the catheter proximal expanded portion 128^{b} partially resides inside the tube distal expanded portion 152^{b}, they are not yet engaged with each other. FIG. 3C shows the catheter proximal expanded portion 128^{b} fully engaged with the tube distal expanded portion 152^{b}, such that the catheter assembly 100^{b} is in an engaged state in FIG. 3C.

The curved shape of the portions of the catheter proximal expanded portion 128 and the tube distal expanded portion 152, namely the portions that include the catheter varying-diameter engagement surface 134 and the tube varying-diameter engagement surface 168, results in full engagement only when a sufficient surface area of engagement surfaces 134, 168 is in frictional contact between the two. In some examples, frictional engagement resulting in an engaged state of the assembly 100 is achieved when both engagement surfaces 134, 168 are fully contacting each other. Partial contact, such as that illustrated in FIG. 3B, will not provide sufficient adherence between the surfaces to allow the catheter 102 and tube 150 to remain engaged with each other. This achieves the effect of a non-adjustable or binary engagement between the catheter 102 and external tube 150.

For example, as shown in FIG. 3B, during insertion of catheter proximal expanded portion 128^{b} into tube distal expanded portion 152^{b}, the catheter varying-diameter engagement surface 134^{b} may not be contacted by any part of the tube varying-diameter engagement surface 168^{b}, or may be contacted by a small portion of the tube varying-diameter engagement surface 168^{b}, such as along a single line of contact (or a narrow area of contact) at the region of tube distal edge 166^{b}. The remainder of the catheter inwardly tapering segment 146^{b}, over which the catheter proximal expanded portion 128^{b} is defined, has an outer diameter that is smaller than the inner diameter of the tube outwardly expanding segment 172^{b}, along which the tube varying-diameter engagement surface 168^{b} is defined, at any other axial position at which both are aligned with each other.

As the catheter proximal expanded portion 128^{b} is further inserted into tube distal expanded portion 152^{b}, until no further axial movement of the catheter 102^{b} and tube 150^{b} is possible, the catheter varying-diameter engagement surface 134^{b} is frictionally engaged with the tube varying-diameter engagement surface 168^{b} along an overlap length L_{O}, as shown in FIG. 3C, such that both surfaces 134, 168 are in contact with each other at any axial position along the length L_{O}. In this position, the catheter assembly 100^{b} is termed to be in an engaged state, maintaining frictional engagement between the catheter 102 and the tube 150 at any axial pull force that does not exceed the maximal non-separating pull force, and enabling disengagement between both when an axially pull force exceeding the threshold separating force is applied thereto.

When the assembly 100 is subjected to an axial pull force that exceeds the threshold separating force, at least a portion of the tube varying-diameter engagement surface 168^{b} will be pulled away from the catheter varying-diameter engagement surface 134^{b}. However, since engagement between the catheter 102 and tube 150 is maintained only as long as a sufficient portion of engagement surfaces 134, 168 are engaged with each other, which in some examples, can be equal to the surface area along the overlap length L_{O}, such partial disengagement may be sufficient to cause the tube 150 to fully disengage from the catheter 102, reverting to the state illustrated, for example, in FIG. 3B and/or FIG. 3A. It is to be understood that any reference herein is a disengaged state of the disengageable catheter assembly 100 refers to the external tube 150 being disconnected and separated from the indwelling catheter 102, such that no portion of the external tube 150 is disposed around or through the indwelling catheter 102 in the disengaged state.

In some examples, catheter varying-diameter engagement surface 134^{b} can be defined along a length equal to the overlap length L_{O}. In some examples, tube varying-diameter engagement surface 168^{b} can be defined along a length equal to the overlap length L_{O}.

In the exemplary implementation of catheter assembly 100^{b}, the outer diameter of catheter inwardly tapering segment 146^{b} is greater than the inner diameter of tube outwardly expanding segment 172^{b} at any axial position of engagement between their engagement surfaces 134^{b}, 168^{b}, such that tube distal expanded portion 152^{b} can be slid over the portion of the catheter proximal expanded portion 128^{b} that includes the engagement surface 134^{b}, to facilitate frictional engagement therebetween. The inner diameter of tube outwardly expanding segment 172^{b} can be equal to or less than the outer diameter of catheter inwardly tapering segment 146^{b} at any axial position of alignment between the two in the engaged state.

In some examples, the inner diameter of tube outwardly expanding segment 172 is slightly smaller than the outer diameter of catheter inwardly tapering segment 146 at any axial position of alignment between the two in the engaged state, meaning that the inner diameter of tube outwardly expanding segment 172 is sufficiently smaller than the outer diameter of catheter inwardly tapering segment 146 at any such axial position to form friction-fit between engagement surfaces 134 and 168, yet not too small so as to allow a user (e.g., the patient or other user) to conveniently manually push the tube distal expanded portion 152 over the appropriate portion of the catheter proximal expanded portion 128, such that, as shown in FIG. 3C, catheter proximal end opening 130^{b} and catheter proximal edge 132^{b} are positioned within tube distal expanded portion 152^{b.}

In some implementations, the catheter expanded portion outer surface 124 comprises the catheter varying-diameter engagement surface 134, and the tube expanded portion inner surface 162 comprises the tube varying-diameter engagement surface 168, as shown for exemplary catheter assembly 100^{b} in FIGS. 3A-3C.

While portions of catheter proximal expanded portion 128 and/or tube distal expanded portion 152 can include surfaces configured to encourage frictional engagement along a length that is longer than L_{O}, the catheter varying-diameter engagement surface 134 and the tube varying-diameter engagement surface 168 are defined as the portions that are configured to be contacted with each other in the engaged state, which can be along the length L_{O} as illustrated, for example, in FIG. 3C.

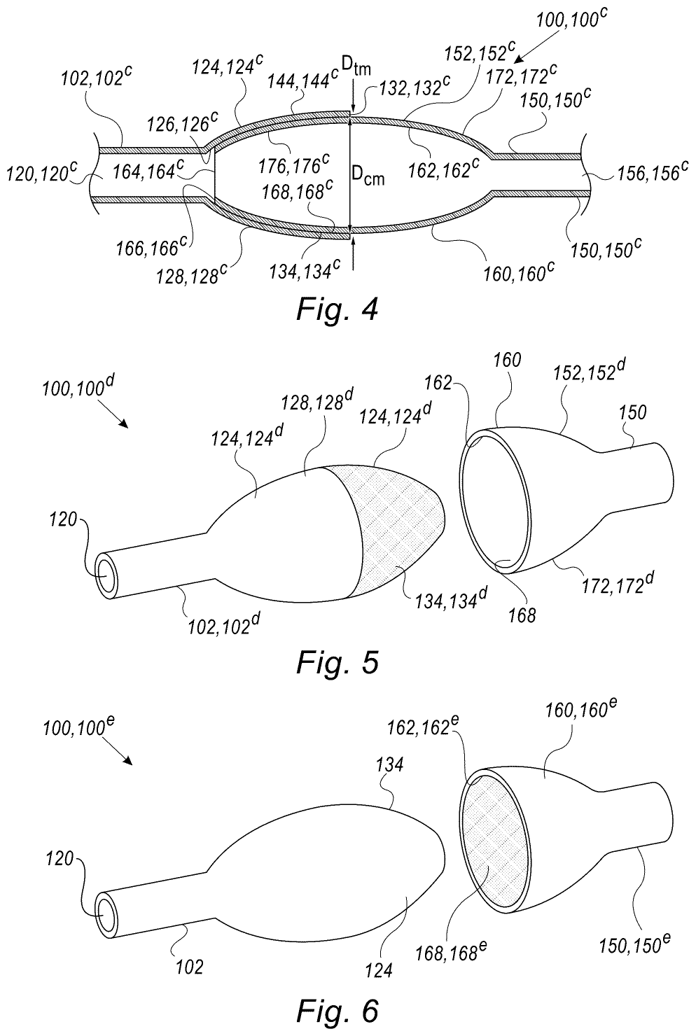

FIG. 4 shows a portion of an exemplary catheter assembly 100^{c}. Catheter assembly 100^{c} is an exemplary implementation of catheter assembly 100, and thus includes all of the features described for catheter assembly 100 and any components thereof throughout the current disclosure, except that while the external tube 150 and indwelling catheter 102 of catheter assembly 100 can have various shapes of expanded portion 128 and 152, such that tube distal expanded portion 152 can either slide over or into catheter proximal expanded portion 128, the catheter proximal expanded portion 128^{c} of indwelling catheter 102^{b} has a prolate hemispheroid shape (i.e., cup-shaped), and the tube distal expanded portion 152^{c} has a prolate spheroid shape (i.e., barrel-shaped), configured to slide into the catheter proximal expanded portion 128^{c} and facilitate frictional engagement therebetween.

In some examples, as illustrated for catheter assembly 100^{c}, the catheter proximal expanded portion 128 is shaped to include a catheter outwardly expanding segment 144 extending proximally from the tubular portion of catheter 102, optionally terminating at the catheter proximal edge 132. The outer diameter of the catheter outwardly expanding segment 144 can gradually increase in a proximal direction from the catheter outer diameter D_{co} of the catheter's tubular portion, to the maximal catheter engagement surface diameter D_{cm}, which can be optionally defined at the catheter proximal edge 132.

In some examples, as illustrated for catheter assembly 100^{c}, the tube distal expanded portion 152 is shaped to include a tube outwardly expanding segment 172 extending distally from the tubular portion of the external tube 150, and a tube inwardly tapering segment 176 extending from the tube outwardly expanding segment 172 and terminating at tube distal edge 166. The inner diameter of the tube outwardly expanding segment 172 can gradually increase in a distal direction from the tube inner diameter Dₜᵢ of the external tube's 150 tubular portion, to the maximal tube engagement surface diameter Dₜₘ. The outer diameter of the tube inwardly tapering segment 176 can gradually decrease in a distal direction from the maximal tube engagement surface diameter Dₜₘ to a smaller diameter at the tube distal edge 166.

In the exemplary implementation of catheter assembly 100^{c}, illustrated in the engaged state, the outer diameter of tube inwardly tapering segment 176^{c} is greater than the inner diameter of catheter outwardly expanding segment 144^{c} at any axial position of engagement between their engagement surfaces 134^{c}, 168^{c}, such that tube distal expanded portion 152^{c} can be slid into the portion of the catheter proximal expanded portion 128^{c} that includes the engagement surface 134^{c}, to facilitate frictional engagement therebetween. The inner diameter of catheter outwardly expanding segment 144^{c} can be equal to or less than the outer diameter of tube inwardly tapering segment 176^{c} at any axial position of alignment between the two in the engaged state.

In some examples, the inner diameter of catheter outwardly expanding segment 144 is slightly smaller than the outer diameter of tube inwardly tapering segment 176 at any axial position of alignment between the two in the engaged state, meaning that the inner diameter of catheter outwardly expanding segment 144 is sufficiently smaller than the outer diameter of tube inwardly tapering segment 176 at any such axial position to form friction-fit between engagement surfaces 134 and 168, yet not too small so as to allow a user (e.g., the patient or other user) to conveniently manually push the tube distal expanded portion 152 into the appropriate portion of the catheter proximal expanded portion 128, such that, as shown in FIG. 4, tube distal end opening 164^{c} and tube distal edge 166^{c} are positioned within catheter proximal expanded portion 128^{c}.

In some implementations, the catheter expanded portion inner surface 126 comprises the catheter varying-diameter engagement surface 134, and the tube expanded portion outer surface 160 comprises the tube varying-diameter engagement surface 168, as shown for exemplary catheter assembly 100^{c} in FIG. 4.

It is to be understood that while catheter assembly 100^{a} is illustrated in FIGS. 2A-2B to include a tube distal expanded portion 152 having a prolate hemispheroid shape configured to slide over a prolate spheroid-shaped catheter proximal expanded portion 128, similar to the configuration described above for catheter assembly 100^{b} and illustrated in FIGS. 3A-3C, this is shown only by way of illustration, and that catheter assembly 100^{a} can be similarly implemented to include an indwelling catheter that has a tube distal expanded portion 152 configured to slide into a catheter proximal expanded portion 128, as described above for catheter assembly 100^{c} and illustrated in FIG. 4, or similar to other exemplary catheter assemblies 100 disclosed herein.

As mentioned above, engagement between catheter 102 and external tube 150 disclosed herein is configured to be performed in a non-adjustable manner, meaning that sufficient overlap between engagement surfaces 134, 168 is required to ensure proper engagement, optionally along a length L_{O} as described above, while a smaller overlap can result in improper engagement that will not meet the threshold axial pull forces described above. While tactile feedback may suffice, as engagement is achieved by manually pushing the tube 150 toward and into or over the catheter 102 until proper engagement may be felt by the user, in some examples, a marking can be added (not shown) to visually indicate when the proper length of overlap between the engagement surfaces 134, 168 is achieved. Such marking can be formed as print or ink-based marking, as a band or strip attached (glued) to a surface of the corresponding catheter 102 and/or tube 150, and the like. The marking can include a geometrical shape, such as in the form of a band, strip, dots, and the like. The marking can include, in some examples, alphanumerical characters and/or strings.

In some examples, a marking can be formed over the catheter expanded portion outer surface 124. In some examples, a marking can be formed over the catheter varying-diameter engagement surface 134. In some examples, a marking can be formed over the tube expanded portion outer surface 160. In some examples, a marking can be formed over the tube varying-diameter engagement surface 168.

In some examples, a marking can be formed over the catheter expanded portion inner surface 126. In some examples, a marking can be formed over the tube expanded portion inner surface 162. In some examples, at least a portion of catheter wall 122 along catheter proximal expanded portion 128 is transparent, to allow visibility of a marking disposed beneath catheter expanded portion outer surface 124, such as along catheter expanded portion inner surface 126 or tube expanded portion outer surface 160. In some examples, at least a portion of tube wall 158 along tube distal expanded portion 152 is transparent, to allow visibility of a marking disposed beneath tube expanded portion outer surface 160, such as along tube expanded portion inner surface 162 or catheter expanded portion outer surface 124. It is to be understood that in some implementations, catheter assembly 100^{a} illustrated in FIGS. 2A-2B, or any other exemplary catheter assembly 100 disclosed herein, can include a marking as described above.

In some implementations, the catheter varying-diameter engagement surface 134 and/or tube varying-diameter engagement surface 168 can be provided as relatively rough or otherwise textured surfaces, to enhance friction engagement in the engaged state. FIG. 5 shows a portion of an exemplary catheter assembly 100^{d}. Catheter assembly 100^{d} is an exemplary implementation of catheter assembly 100, and thus includes all of the features described for catheter assembly 100 and any components thereof throughout the current disclosure, except that while catheter varying-diameter engagement surface 134 of catheter assembly 100 can have any surface roughness, including textured or smooth surfaces, the catheter varying-diameter engagement surface 134^{d} of catheter assembly 100^{e} can be provided as a relatively rough surface to increase frictional engagement in the engaged state.

In some implementations, catheter varying-diameter engagement surface 134^{d} can be rougher than at least one other surface of the indwelling catheter 102^{d}. In some examples, when catheter varying-diameter engagement surface 134 is comprised in catheter expanded portion outer surface 124, it can be rougher than catheter expanded portion inner surface 126. In some examples, when catheter varying-diameter engagement surface 134 is comprised in catheter expanded portion inner surface 126, it can be rougher than catheter expanded portion outer surface 124. In some implementations, the catheter varying-diameter engagement surface 134 extends along a length L_{O}, which is rougher than the remainder of the corresponding surface extending distally from an end of length L_{O}.

In the example illustrated in FIG. 5, catheter varying-diameter engagement surface 134^{d}, which is formed as a portion of catheter expanded portion outer surface 124^{d} along catheter inwardly tapering segment 144, can be rougher than the remainder of catheter expanded portion outer surface 124^{d}, such as along catheter outwardly expanding segment 144, as well as rougher than the outer surface of the tubular portion of catheter 102^{d}. Such a configuration can be advantageous for increasing friction engagement in the engaged state, while leaving the rest of the outer surface of catheter 102^{d} relatively smooth to refrain from irritating the skin at the body point of entry 98 and/or any other organs within the patient's body.

Surface roughness is a component of surface texture. It is quantified by the deviations in the direction of the normal vector of a real surface from its ideal form. If these deviations are large, the surface is considered rough, and if they are small, the surface is considered smooth. Therefore, the term “smooth”, as used herein refers to a surface having minor deviations in the direction of the normal vector of a real surface from its ideal form. Smooth surfaces are substantially unitary/continuous surfaces, free from irregular voids. The term “smooth” is not intended to be limited to the narrow meaning of a substantially planar surface devoid of surface irregularities.

Surface roughness is typically calculated by a method termed “Ra” or roughness average, which represents the arithmetic average of a set of individual measurements of surfaces peaks and valleys (e.g., normal vectors), relative to a mean line (e.g., a real surface), wherein low Ra values represents smooth surfaces. The Ra value for any rough surface disclosed herein is selected to provide frictional force, in the engaged state, that allows tube 150 to be release from catheter 102 when a pull force exceed the threshold separating force as described hereinabove.

FIG. 6 shows a portion of an exemplary catheter assembly 100^{e}. Catheter assembly 100^{e} is an exemplary implementation of catheter assembly 100, and thus includes all of the features described for catheter assembly 100 and any components thereof throughout the current disclosure, except that while tube varying-diameter engagement surface 168 of catheter assembly 100 can have any surface roughness, including textured or smooth surfaces, the tube varying-diameter engagement surface 168^{e} of catheter assembly 100^{e} can be provided as a relatively rough surface to increase frictional engagement in the engaged state.

In some implementations, tube varying-diameter engagement surface 168^{e} can be rougher than at least one other surface of the external tube 150^{e}. In some examples, when tube varying-diameter engagement surface 168 is comprised in tube expanded portion outer surface 160, it can be rougher than tube expanded portion inner surface 162. In some examples, when tube engagement surface 168 is comprised in tube expanded portion inner surface 162, it can be rougher than tube expanded portion outer surface 160. In some implementations, the tube varying-diameter engagement surface 168 extends along a length L_{O}, which is rougher than the remainder of the corresponding surface extending proximally from an end of length L_{O}. In the example illustrated in FIG. 6, tube varying-diameter engagement surface 168^{e}, which is formed as a portion of tube expanded portion inner surface 162^{e}, can be rougher than the inner surface along the remainder to external tube 150^{d}.

While a rough or textured catheter varying-diameter engagement surface 134^{d} is shown in FIG. 5 as part of catheter expanded portion outer surface 124^{d}, it is to be understood that this is shown by way of illustration and not limitation, and that a rough catheter varying-diameter engagement surface 134 can be similarly comprises in catheter expanded portion inner surface 126. While a rough or textured tube varying-diameter engagement surface 168^{e} is shown in FIG. 6 as part of tube expanded portion inner surface 162^{e}, it is to be understood that this is shown by way of illustration and not limitation, and that a rough tube varying-diameter engagement surface 168 can be similarly comprises in tube expanded portion inner surface 162. While catheter varying-diameter engagement surface 134^{d} and tube varying-diameter engagement surface 168^{e} are illustrated to have higher surface roughness than the remainder of the surfaces they are comprised in, such as respective catheter expanded portion outer surface 124^{d} and tube expanded portion inner surface 162^{e}, it is to be understood that this is shown by way of illustration and not limitation, and that the entire surfaces in which catheter varying-diameter engagement surface 134 and/or tube varying-diameter engagement surface 168, such as any of the surfaces 124, 126, 160, 162, can be provided with a relatively high roughness along the entire lengths of the corresponding catheter 102 or tube 150.

In some examples, catheter varying-diameter engagement surface 134 can be provided with greater surface roughness than that of tube varying-diameter engagement surface 168. In some examples, tube varying-diameter engagement surface 168 can be provided with greater surface roughness than that of varying-diameter catheter engagement surface 134. In some examples, catheter varying-diameter engagement surface 134 and tube varying-diameter engagement surface 168 can have a similar roughness. While a rough catheter varying-diameter engagement surface 134^{d} and a rough tube varying-diameter engagement surface 168^{e} are separately illustrated in FIGS. 5 and 6, respectively, it is to be understood that they can be combined such that both engagement surfaces 134, 168 of a catheter assembly 100 can be provided with high surface roughness.

It is to be understood that while catheter assemblies 100^{d} and 100^{e} are illustrated in FIGS. 5 and 6, respectively, to include a tube distal expanded portion 152 having a prolate hemispheroid shape configured to be disposed over a prolate spheroid-shaped catheter proximal expanded portion 128, similar to the configuration described above for catheter assembly 100^{b} and illustrated in FIGS. 3A-3C, this is shown only by way of illustration, and that catheter assemblies 100^{d} and 100^{e}, as well as combinations thereof as described above, can be similarly implemented to include an indwelling catheter that has a tube distal expanded portion 152 configured to slide into a catheter proximal expanded portion 128, as described above for catheter assembly 100^{c} and illustrated in FIG. 4, in which case rough catheter varying-diameter engagement surface 134 can be comprised in catheter expanded portion inner surfaces 126, and rough tube varying-diameter engagement surface 168 can be comprised in tube expanded portion outer surface 160. It is to be understood that in some implementations, any of varying-diameter engagement surfaces 134^{a}, 168^{a} of catheter assembly 100^{a} illustrated in FIGS. 2A-2B can be provided with high surface roughness as described above.

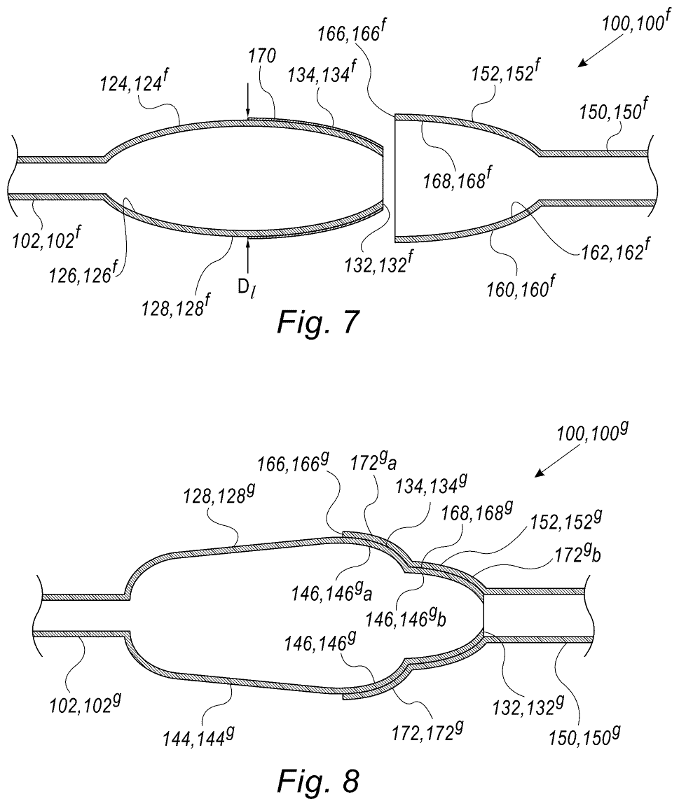

FIG. 7 shows a portion of an exemplary catheter assembly 100^{f}. Catheter assembly 100^{f} is an exemplary implementation of catheter assembly 100, and thus includes all of the features described for catheter assembly 100 and any components thereof throughout the current disclosure, except that while any varying-diameter engagement surface 134, 168 of a catheter assembly 100 can formed as part of outer or inner surfaces of the catheter 102 and/or tube 150, or as a surface of a layer attached to outer or inner surfaces of the catheter 102 and/or tube 150, catheter assembly 100^{f} further comprises at least one friction layer 170 disposed over (e.g., attached to or coated over) at least one of the catheter 102^{f} and/or tube 150^{f}, wherein the at least one friction layer 170 comprises varying-diameter engagement surface(s) 134^{f} and/or 168^{f}.

In the example illustrated in FIG. 7, the indwelling catheter 102^{f} includes a friction layer 170 attached to catheter expanded portion outer surface 124^{f} along catheter inwardly extending tapering segment 146. The friction layer 170 can optionally extend distally from catheter proximal edge 132^{f}. In the illustrated example, catheter varying-diameter engagement surface 134^{f} is not formed as a portion of catheter expanded portion outer surface 124^{f}, but is rather formed by or comprised in the friction layer 170 which is attached to and covers a portion of catheter expanded portion outer surface 124^{f}. Thus, tube varying-diameter engagement surface 168^{f} is configured to frictionally engage with catheter varying-diameter engagement surface 134^{f}, which is defined as an outer surface of the friction layer 170 to provide the desired frictional force in the engaged state.

In some examples, friction layer 170 is directly coated over the corresponding surface of the catheter 102 and/or tube 150. In some examples, friction layer 170 is formed as a separate component attached to the corresponding surface of the catheter 102 and/or tube 150, such as by being provided as a band or strip glued or otherwise affixed thereto, or by being provided as a ring, band or sleeve slid over and frictionally engaged or press fit over the corresponding surface of the catheter 102 and/or tube 150. In some examples, the friction layer 170 is made of a material which is different from the material of the corresponding catheter wall 122 and/or tube wall 158 it is attached to.

In some examples, a friction layer 170 is attached to the catheter expanded portion outer surface such that the catheter varying-diameter engagement surface 134 is an outer surface of the friction layer 170, as illustrated in FIG. 7 for example. In some examples, a friction layer 170 is attached to the catheter expanded portion inner surface 126, such that the catheter varying-diameter engagement surface 134 is an inner surface of the friction layer 170. In some examples, a friction layer 170 is attached to the tube expanded portion outer surface 160, such that the tube varying-diameter engagement surface 168 is an outer surface of the friction layer 170. In some examples, a friction layer 170 is attached to the tube expanded portion inner surface 162, such that the tube varying-diameter engagement surface 168 is an inner surface of the friction layer 170.

It is to be understood that while catheter assembly 100^{f} is illustrated in FIG. 7 to include a tube distal expanded portion 152 having a prolate hemispheroid shape configured to slide over a prolate spheroid-shaped catheter proximal expanded portion 128, similar to the configuration described above for catheter assembly 100^{b} and illustrated in FIGS. 3A-3C, this is shown only by way of illustration, and that catheter assembly 100^{f} can be similarly implemented to include an indwelling catheter that has a tube distal expanded portion 152 configured to slide into a catheter proximal expanded portion 128, as described above for catheter assembly 100^{c} and illustrated in FIG. 4, in which case the friction layer 170 can be optionally attached to catheter expanded portion inner surface 126.

While the friction layer 170 of catheter assembly 100^{f} is illustrated in FIG. 7 to be attached to the indwelling catheter 102^{f}, it is to be understood that friction layer 170 can be similarly attached to the inner 162 or outer 160 surface of tube distal expanded portion 152. Moreover, it is to be understood that while only indwelling catheter 102^{f} is shown in FIG. 7 to include a friction layer 170, this is shown by way of illustration, and that in some implementations, both the catheter 102 and the tube 150 can include corresponding friction layers 170 attached to surfaces thereof, such that a friction layer 170 of catheter 102 is configured to frictionally engage with the friction layer 170 of tube 150. For example, catheter 102 can include a friction layer 170 attached to the catheter expanded portion outer surface 124, configured to frictionally engage a corresponding friction layer 170 attached to the tube expanded portion inner surface. Similarly, catheter 102 can include a friction layer 170 attached to the catheter expanded portion inner surface 126, configured to frictionally engage a corresponding friction layer 170 attached to the tube expanded portion outer surface 160.

It is to be understood that in some implementations, a friction layer 170 can further include a marking as described above. It is to be understood that in some implementations, catheter assembly 100^{a} illustrated in FIGS. 2A-2B can include one or more friction layers 170 as described above.

FIG. 8 shows a portion of an exemplary catheter assembly 1008. Catheter assembly 100^{g} is an exemplary implementation of catheter assembly 100, and thus includes all of the features described for catheter assembly 100 and any components thereof throughout the current disclosure, except that the indwelling tube 102^{g} defines a multi-segmented catheter varying-diameter engagement surface 134^{g}, and the external tube 150^{g} defines a multi-segmented tube varying-diameter engagement surface 168^{g}. In the illustrated example, catheter proximal expanded portion 128^{g} is shown to include a curved catheter outwardly expanding segment 144^{g}, and a multi-segmented catheter inwardly tapering segment 146^{g} comprising a first curved catheter inwardly tapering segment 146^{g}a extending proximally from the catheter outwardly expanding segment 144^{g}, and a second curved catheter inwardly tapering segment 146^{g}b extending proximally from the first curved catheter inwardly tapering segment 146^{g}a to catheter proximal edge 132^{g}. As shown, the curvatures of the first and second catheter inwardly tapering segments 146^{g}a and 146^{g}b can be different from each other. In some examples, the radius of curvature of the first catheter inwardly tapering segment 146^{g}a is smaller than that of the second catheter inwardly tapering segment 146^{g}b.

The tube distal expanded portion 152^{g} is shown to include a multi-segmented tube outwardly expanding segment 172^{g}, comprising a first curved tube outwardly expanding segment 172^{g}a extending proximally from the tube distal edge 166^{g}, and a second curved tube outwardly expanding segment 172^{g}b extending proximally from the first curved tube outwardly expanding segment 172^{g}a toward the tubular portion of external tube 150^{g}. As shown, the curvatures of the first and second tube outwardly expanding segments 172^{g}a and 172^{g}b can be different from each other. In some examples, the radius of curvature of the first tube outwardly expanding segment 172^{g}a is smaller than that of the second first tube outwardly expanding segment 172^{g}b.

In the illustrated example, the catheter varying-diameter engagement surface 134^{g} is defined over the outer surface 124 of multi-segmented catheter inwardly tapering segment 146^{g}, and the tube varying-diameter engagement surface 168^{g} is defined along the inner surface 162 of multi-segmented tube outwardly expanding segment 172^{g}, wherein the inner diameter of the first and second tube outwardly expanding segments 172^{g}a and 172^{g}b can be similar to or less than the outer diameter of the first and second catheter inwardly tapering segments 146^{g}a and 146^{g}b, respectively, at each axial position of engagement between the engagement surfaces 134^{g}, 168^{g}.

While two segments are illustrated for each of the catheter inwardly tapering segment 146^{g} and the tube outwardly expanding segment 172^{g}, it is to be understood that any other number of segments is contemplated. While catheter assembly 100^{g} is shown to include a multi-segmented tube outwardly expanding segment 172^{g} configured to slide over a multi-segmented catheter inwardly tapering segment 146^{g}, it is to be understood that in some examples, a catheter assembly 100 can include a multi-segmented tube inwardly tapering segment 176 that can extend distally from a tube outwardly expanding segment, which is configured to slide into a corresponding multi-segmented catheter outwardly expanding segment 144 (example not shown). In such examples, the catheter varying-diameter engagement surface 134 can be defined along the inner surface 126 of the multi-segmented catheter outwardly expanding segment 144, and the tube varying-diameter engagement surface can be defined over the outer surface 160 of the multi-segmented tube inwardly tapering segment 176.

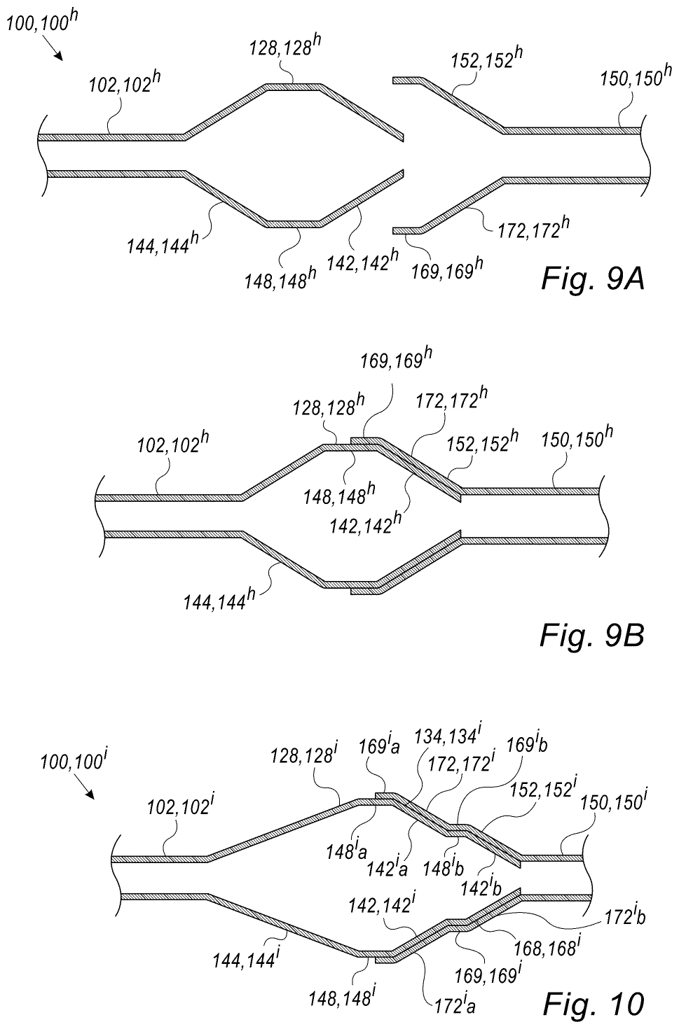

FIGS. 9A and 9B are cross-sectional views of a portion of an exemplary disengageable catheter assembly 100^{h} shown in a disengaged state and an engaged state, respectively. Catheter assembly 100^{h} is an exemplary implementation of catheter assembly 100, and may be implemented according to any example described above with respect to catheter assembly 100^{b}, except that while the catheter varying-diameter engagement surface 134^{b} and tube varying-diameter engagement surface 168^{b} are illustrated to extend along curved segments of the catheter proximal expanded portion 128^{b} and the tube distal expanded portion 152^{b}, respectively, the catheter varying-diameter engagement surface 134^{h} and tube varying-diameter engagement surface 168^{h} of catheter assembly 100^{h} extend along angled segments of the catheter proximal expanded portion 128^{h} and the tube distal expanded portion 152^{h}, respectively.

In the illustrated example, the catheter proximal expanded portion 128^{h} is shown to include a frustoconical catheter outwardly expanding segment 144^{h}, and a frustoconical catheter inwardly expanding segment 144^{h}. The tube distal expanded portion 152^{h} is shown to include a frustoconical tube outwardly expanding segment 172^{h}.

In some examples, the catheter proximal expanded portion 128 can include at least one catheter flat 148, shaped as a cylindrical section having a uniform diameter. In some examples, the tube distal expanded portion 152 can include at least one tube flat 169, shaped as a cylindrical section having a uniform diameter. In the illustrated examples, the catheter proximal expanded portion 128^{h} is shown to include a catheter flat 148^{h} extending between the catheter outwardly expanding segment 144^{h} and the catheter inwardly expanding segment 144^{h}, and the tube distal expanded portion 152^{h} is shown to include a tube flat 169^{h} extending distally from the tube outwardly expanding segment 172^{h} to the tube distal edge 166.

The catheter varying-diameter engagement surface 134^{h} can be defined over the outer surface 124 of at least a portion of the catheter flat 148^{h} and at least a portion of the catheter inwardly expanding segment 144^{h}. The tube varying-diameter engagement surface 168^{h} can be defined along the inner surface 162 of at least a portion of the tube flat 169^{h} and at least a portion of the tube outwardly expanding segment 172^{h}. The inner diameter of the tube flat 169^{h} and the tube outwardly expanding segment 172^{h} can be similar to or less than the catheter flat 148^{h} and the catheter inwardly expanding segment 144^{h}, respectively, at each axial position of engagement between the engagement surfaces 134^{h}, 168^{h}.

While frustoconical sections of catheter proximal expanded portion 128^{h} and tube distal expanded portion 152^{h} are shown in the illustrated example in combination with a catheter flat 148^{h} and a tube flat 169^{h}, it is to be understood that this is shown by way of illustration and not limitation, and that catheter proximal expanded portion 128 and tube distal expanded portion 152 can include frustoconical segments without including flats 148 or 169, or can include flats 148, 169 next to otherwise-shaped expanding and/or tapering segments.

While catheter assembly 100^{h} is shown to include a frustoconical tube outwardly expanding segment 172^{h} configured to slide over a frustoconical catheter inwardly tapering segment 146^{h}, it is to be understood that in some examples, a catheter assembly 100 can include a frustoconical tube inwardly tapering segment 176, distal to a frustoconical tube outwardly expanding segment 172, which is configured to slide into a corresponding frustoconical catheter outwardly expanding segment 144 (example not shown). In such examples, the catheter varying-diameter engagement surface 134 can be defined along the inner surface 126 of at least a portion of the frustoconical catheter outwardly expanding segment 144, and the tube varying-diameter engagement surface 168 can be defined over the outer surface 160 of the at least a portion of the frustoconical tube inwardly tapering segment 176.

FIG. 10 is a cross-sectional views of a portion of an exemplary disengageable catheter assembly 100ⁱ. Catheter assembly 100ⁱ is an exemplary implementation of catheter assembly 100, and may be implemented according to any example described above with respect to catheter assembly 100^{g}, except that while the catheter varying-diameter engagement surface 134^{g} and tube varying-diameter engagement surface 168^{g} are illustrated to extend along multi-segmented sections of the catheter proximal expanded portion 128^{g} and the tube distal expanded portion 152^{g}, formed to include curved segments as shown, for example, in FIG. 8, the catheter varying-diameter engagement surface 134ⁱ and tube varying-diameter engagement surface 168ⁱ of catheter assembly 100ⁱ extend along multi-segmented sections of the catheter proximal expanded portion 128ⁱ and the tube distal expanded portion 152ⁱ, formed to include frustoconical segments, and optionally one or more flats such as catheter flats 148ⁱ and tube flats 169ⁱ.

In the illustrated example, catheter proximal expanded portion 128ⁱ is shown to include a frustoconical catheter outwardly expanding segment 144ⁱ, and a multi-segmented catheter inwardly tapering segment 146ⁱ comprising a first frustoconical catheter inwardly tapering segment 146ⁱa and a second frustoconical catheter inwardly tapering segment 146ⁱb terminating at catheter proximal edge 132ⁱ. The tube distal expanded portion 152ⁱ is shown to include a multi-segmented tube outwardly expanding segment 172ⁱ, comprising a first curved tube outwardly expanding segment 172ⁱa, and a second frustoconical tube outwardly expanding segment 172ⁱb proximal to the first frustoconical tube outwardly expanding segment 172^{g}a.

A first catheter flat 148ⁱa is also shown to extend between the frustoconical catheter outwardly expanding segment 144ⁱ and first frustoconical catheter inwardly tapering segment 146ⁱa, and a second catheter flat 148ⁱb extends between the first and second frustoconical catheter inwardly tapering segments 146ⁱa and 146ⁱb. Similarly, a first tube flat 169ⁱa is shown to extend from the tube distal edge 166ⁱa to the first curved tube outwardly expanding segment 172ⁱa, and a second distal edge 166ⁱb extends between the first and second frustoconical tube outwardly expanding segments 172ⁱa and 172ⁱb.

The catheter varying-diameter engagement surface 134ⁱ can be defined over the outer surface 124 of the multi-segmented catheter inwardly tapering segment 146ⁱ, optionally including along the second catheter flat 148ⁱb and at least a portion of the first catheter flat 148ⁱa. The tube varying-diameter engagement surface 168ⁱ can be defined along the inner surface 162 of at least a portion of multi-segmented tube outwardly expanding segment 172ⁱ, optionally including along both tube flats 169ⁱ. The inner diameter of first and second tube outwardly expanding segments 172ⁱa and 172ⁱb, as well as first and second tube flats 169ⁱa and 169ⁱb, can be similar to or less than the outer diameter of the first and second catheter inwardly tapering segments 146ⁱa and 146ⁱb, as well as first and second catheter flats 148ⁱa and 148ⁱb, respectively, at each axial position of engagement between the engagement surfaces 134ⁱ, 168ⁱ.

While two frustoconical segments and two flats are illustrated for each of the catheter inwardly tapering segment 146ⁱ and the tube outwardly expanding segment 172ⁱ, it is to be understood that any other number of segments and/or flats is contemplated. While catheter assembly 100ⁱ is shown to include a multi-segmented tube outwardly expanding segment 172ⁱ configured to slide over a multi-segmented catheter inwardly tapering segment 146ⁱ, it is to be understood that in some examples, a catheter assembly 100 can include a multi-segmented tube inwardly tapering segment 176 that can extend distally from a tube outwardly expanding segment, which is configured to slide into a corresponding multi-segmented catheter outwardly expanding segment 144 (example not shown). In such examples, the catheter varying-diameter engagement surface 134 can be defined along the inner surface 126 of the multi-segmented catheter outwardly expanding segment 144, and the tube varying-diameter engagement surface can be defined over the outer surface 160 of the multi-segmented tube inwardly tapering segment 176.

While multi-segmented sections of catheter proximal expanded portion 128ⁱ and tube distal expanded portion 152ⁱ are shown in the illustrated example in combination with catheter flats 148ⁱ and tube flats 169ⁱ, it is to be understood that this is shown by way of illustration and not limitation, and that multi-segmented catheter proximal expanded portion 128 and multi-segmented tube distal expanded portion 152 can include frustoconical segments without including flats 148 or 169, or can include flats 148, 169 next to otherwise-shaped multi-segmented expanding and/or tapering segments.

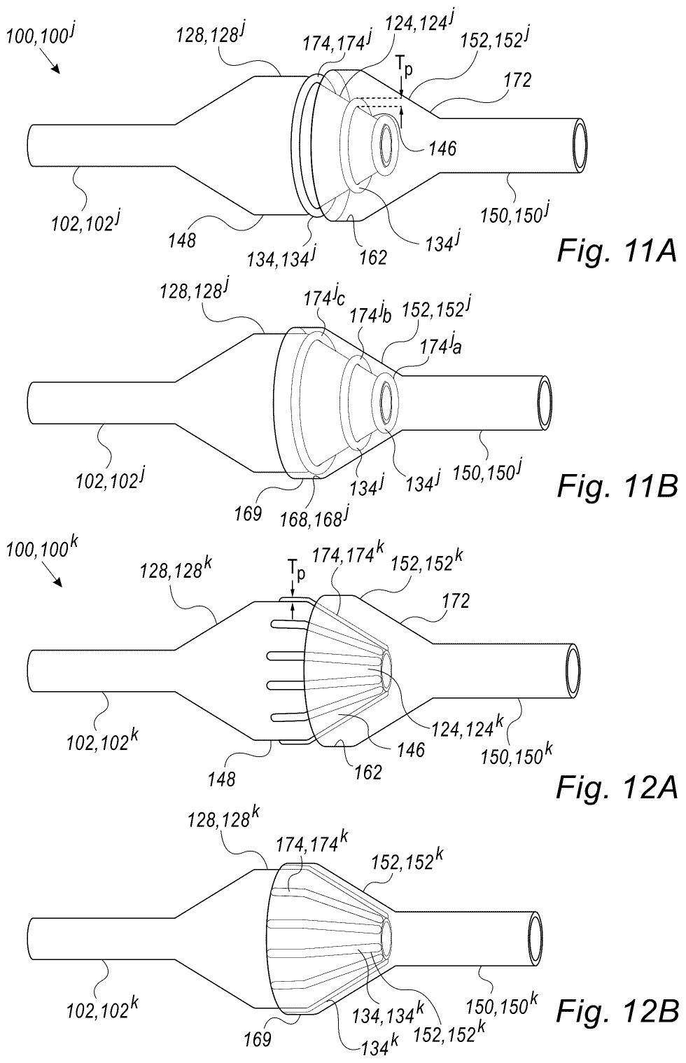

FIGS. 11A and 11B are side views in perspective of a portion of an exemplary disengageable catheter assembly 100^{j} shown in a disengaged state and an engaged state, respectively. Catheter assembly 100^{j} is an exemplary implementation of catheter assembly 100, and thus includes all of the features described for catheter assembly 100 and any components thereof throughout the current disclosure, except that while any engagement surface 134, 168 of catheter assembly 100 can be formed as part of outer or inner surfaces of catheter proximal expanded portion 128 or tube distal expanded portion 152, or as a part or component attached to and/or protruding from outer or inner surfaces of catheter proximal expanded portion 128 or tube distal expanded portion 152, catheter assembly 100^{j} further comprises a plurality of protrusions 174 extending from at least one of the catheter proximal expanded portion 128^{j} or tube distal expanded portion 152^{j}, wherein the protrusions 174 comprise the engagement surface(s) 134^{j} and/or 168^{j}.

In the example illustrated in FIGS. 11A-11B, the catheter proximal expanded portion 128^{j} is shown to include a plurality of circumferential protrusions 174^{j}, axially spaced from each other, each having a protrusion thickness T_{P}. In the illustrated example, catheter varying-diameter engagement surface 134^{j} is not formed as a portion of catheter expanded portion outer surface 124^{j}, but is rather defined by the combined outer surfaces of circumferential protrusions 174^{j}. In such a case, tube varying-diameter engagement surface 168^{j} is defined as the portions of tube expanded portion inner surface 162^{j} that come in contact with circumferential protrusions 174^{j} in the expanded state shown in FIG. 11B, configured to frictionally engage with catheter engagement surface 134^{j}, and more specifically, with the outer surface portions of circumferential protrusions 174^{j}. The inner diameter of the tube outwardly expanding segment 172^{j} can be similar to or less than the combined outer diameter of the catheter inwardly tapering segment 146^{j} and twice the protrusion thickness T_{P}, at each axial position of engagement between the engagement surfaces 134^{j}, 168^{j} (i.e., at each point of contact between the tube expanded portion inner surface 162^{j} with a corresponding circumferential protrusion 174^{j}) in the expanded state.

FIGS. 12A and 12B are side views in perspective of a portion of an exemplary disengageable catheter assembly 100^{k} shown in a disengaged state and an engaged state, respectively. Catheter assembly 100^{k} is an exemplary implementation of catheter assembly 100, and can be similar to any examples described herein for catheter assembly 100^{j}, except that while catheter 100^{j} includes a plurality of circumferential protrusions 174^{j} axially spaced from each other, catheter assembly 100^{k} comprises a plurality of axial protrusions 174^{j}, circumferentially spaced from each other, each having a similar protrusion thickness T_{P}. The catheter varying-diameter engagement surface 134^{k} can be defined, in the illustrated example, by the combined outer surfaces of axial protrusions 174^{k}, in which case the tube varying-diameter engagement surface 168^{k} can be defined by the portions of tube expanded portion inner surface 162^{k} that come in contact with axial protrusions 174^{k} in the expanded state shown in FIG. 12B, configured to frictionally engage with catheter engagement surface 134^{k}, and more specifically, with the outer surface portions of circumferential protrusions 174^{k}. The inner diameter of the tube outwardly expanding segment 172^{k} can be similar to or less than the combined outer diameter of the catheter inwardly tapering segment 146^{k} and twice the protrusion thickness T_{P}, at each axial position of engagement between the engagement surfaces 134^{k}, 168^{k} (i.e., at each point of contact between the tube expanded portion inner surface 162^{k} with the axial protrusions 174^{k}) in the expanded state.

While circumferential protrusions 174^{j} or helical protrusions 174^{k} are illustrated, it is to be understood that any other shape is contemplated. For example, protrusions 174 can extend at an angle over and around portions of catheter proximal expanded portion 128 and/or tube distal expanded portion 152. Similarly, catheter assembly 100 is not limited to include a plurality of protrusions, but may also include a single protrusion 174 shaped to defined a varying-diameter engagement surface. For example, a catheter assembly 100 can include a single protrusion 174 helically wrapped around catheter proximal expanded portion 128 and/or tube distal expanded portion 152, optionally defining more than one turn. Any reference to protrusions 174 in the plural form, similarly refers to examples in which a single protrusion is included. Any reference to protrusions 174 without a superscript, can refer to circumferential protrusion 174^{j}, axial protrusions 174^{k}, or otherwise-shaped protrusions.

In some examples, protrusions 174 are integrally formed with catheter wall 122 and/or tube wall 158, for example by mold-injection procedures in which a mold is used to form the indwelling catheter 102 and/or external catheter 150, the mold formed to define such protrusions 174 as well. In some examples, protrusions 174 are formed as separate components attached to the corresponding surface of the catheter proximal expanded portion 128 and/or tube distal expanded portion 152. For example, circumferential protrusion 174′can be provided as a bands or rings, and axial protrusions 174^{k} can be provided as elongated tubes or strips, glued or otherwise affixed to catheter proximal expanded portion 128 and/or tube distal expanded portion 152. Circumferential protrusion 174^{j} can be optionally provided as rings or bands slid over and frictionally engaged or press fit over the corresponding surface of the catheter proximal expanded portion 128^{j} and/or tube distal expanded portion 152^{j}, such as in the form of O-rings tightly fit over the corresponding catheter proximal expanded portion 128^{j} and/or tube distal expanded portion 152^{j}. In some examples, the protrusions 174 are made of a material which is different from the material of the corresponding catheter wall 122 and/or tube wall 158 they are attached to.

In some examples, protrusions 174 protrude outwardly from the catheter expanded portion outer surface 124, such that the catheter varying-diameter engagement surface 134 is defined by the plurality of outwardly-facing surfaces of the protrusions 174, as illustrated in FIGS. 11A-12B for example. In some examples, protrusions 174 protrude inwardly from the catheter expanded portion inner surface 126, such that the catheter varying-diameter engagement surface 134 is defined by the inwardly-facing surfaces of the protrusions 174. In some examples, protrusions 174 protrude outwardly from the tube expanded portion outer surface 160, such that the tube varying-diameter engagement surface 168 is defined by the outwardly-facing surfaces of the protrusions 174. In some examples, protrusions 174 protrude inwardly from the tube expanded portion inner surface 162, such that the tube varying-diameter engagement surface 168 is defined by the inwardly-facing surfaces of the protrusions 174.

While catheter proximal expanded portion 128 and tube distal expanded portion 152 are illustrated in FIGS. 11A-12B to include frustoconical sections in combination with catheter flats 148 and a tube flats 169, it is to be understood that this is shown by way of illustration and not limitation, and that catheter proximal expanded portion 128 and tube distal expanded portion 152 formed to include one or more protrusions 174, can include otherwise-shaped expanding and/or tapering segments, and can be provided with or without flats 148, 169.

While each of the catheter assemblies 100^{j} and 100^{k} is shown to include a tube outwardly expanding segment 172 configured to slide over a catheter inwardly tapering segment 146, it is to be understood that in some examples, a catheter assembly 100 provided with one or more protrusions 174 can include a tube inwardly tapering segment 176, distal to a tube outwardly expanding segment 172, which is configured to slide into a corresponding catheter outwardly expanding segment 144 (example not shown). In such examples, one or more protrusions 174 can extend outwardly from the tube expanded portion outer surface 160, for example.

It is to be understood that in some implementations, at least one protrusion 174 can serve as a marking as described above. It is to be understood that in some implementations, catheter assembly 100^{a} illustrated in FIGS. 2A-2B can include protrusions 174 as described above.

As mentioned above, catheter distal portion 104 can include an anchoring mechanism 110 that include a pigtail 112. An anchoring mechanism 110 configured to transition the catheter distal portion 104 from a relatively straight or compacted configuration, to an expanded or curled configuration of a pigtail 112, can include a flexible tensioning member 140 extending between the catheter proximal end portion 128 and catheter distal portion 104. FIGS. 13A-13D show different stages of utilizing an anchoring mechanism 110^{l} of catheter assembly 100^{l}, which is an exemplary implementation of disengageable catheter assembly 100, and thus includes all of the features described for disengageable catheter assembly 100 throughout the current disclosure, except that while the indwelling catheter 102 of disengageable catheter assembly 100 can include different types of anchoring mechanisms 110, disengageable catheter assembly 100^{l} includes an indwelling catheter which is implemented as a pigtail catheter 102^{l}, provided with an anchoring mechanism 110^{l} that comprises a pigtail 112 configuration that can transition between compacted and expanded or curled configurations via flexible tensioning member 140.

FIG. 13A shows the indwelling catheter 102^{l} with catheter distal portion 104^{l} in a compacted or unexpanded state, relatively straightened for convenient insertion and implantation in a patient's body. The indwelling catheter 102^{l} comprises a flexible tensioning member 140, such as a cord, suture, string and the like, which is affixed to a proximal portion of the catheter 102^{l} on one end thereof, such as to an inner surface of catheter 102^{l} within catheter proximal expanded portion 128^{l} or at some proximity thereto, while the opposite end of the flexible tensioning member 140 extends through (or otherwise attached to) the catheter distal portion 104. In the illustrated example, the flexible tensioning member 140 is shown to extend through a draw port 138a formed in catheter wall 122^{l}, out of catheter 102^{l} toward and through another draw port 138b formed in catheter wall 122^{l}, spaced away from draw port 138a along catheter distal portion 104^{l}. Flexible tensioning member 140 extends through draw port 138b back into catheter 102^{l}, and then extends along catheter 102^{l} back toward catheter proximal end portion 128^{l}, terminating at a free tail extending out of catheter 102^{l}, optionally exiting from catheter proximal end opening 130^{l}.

Upon inserting indwelling catheter 102^{l} into the patient's body, the free end of flexible tensioning member 140 that protrudes out of catheter proximal expanded portion 128^{l} can be pulled tight, causing a loop to be formed as shown in FIGS. 13B and 13C. The loop formation of pigtail 112 is configured to maintain the indwelling catheter 102^{l} within the patient's body, and in particular, to maintain the catheter distal portion 104^{l} within the organ 90.

The flexible tensioning member 140 must maintain its tension in order for pigtail 112 to remain in its curled configuration in organ 90. Therefore, flexible tensioning member 140 should be secured after it has been pulled tight. In some examples, this is achieved by tying the free end of flexible tensioning member 140 to a fixed object, such as tying it over a region of catheter 102^{l} which is slightly distal to catheter proximal expanded portion 128^{l} as illustrated in FIG. 13C. While some conventional indwelling catheters, such as an indwelling catheter 10 illustrated in FIG. 1, can include an anchoring mechanism 12 by which a flexible tensioning member is similarly pulled to assume a curled pigtail shape of the distal end portion 14, the flexible tensioning member of such catheters 10 extends over a longer portion of the catheter 10, having a relatively longer length extending away from the patient's body, with the free end of the flexible tensioning member extending out of, and optionally tied to, a proximal end portion 16 that can optionally include a connector 20 or hub over which it is tied. In contrast, the portion of indwelling catheter 102^{l} of catheter assembly 100^{l} includes a relatively shorter exterior portion extending out of the patient's body, from which the flexible tensioning member 140 extend and to which it should be secured.

As shown in FIG. 13D, when external tube 150^{l} is frictionally engaged with catheter 102^{l}, the tube distal expanded portion 152^{l} can contact and optionally slide over a portion of the flexible tensioning member 140 extending over catheter proximal expanded portion 128^{l}. It may be of importance, in some implementations, to secure the flexible tensioning member 140 to catheter proximal expanded portion 128 such that the flexible tensioning member 140 maintains tension to keep the pigtail 112 in a curled configuration when external tube 150^{l} is moved to engage with catheter 102^{l}, as well as when external tube 150^{l} is pulled away to disengage from catheter 102^{l}. This is important so that if the tube 150^{l} is pulled away from catheter 102^{l}, for example when a pulling force exceeds the threshold separating force, the pigtail 112 remains anchored in the organ 90 (e.g., the patient's kidney), to prevent accidental dislodgment thereof if otherwise it knot is released and the catheter distal portion 104 is straightened. In some examples, the knot 142 is formed distal to the tube distal edge 166 when in the engaged state.

Flexible tensioning member 140 can extend through catheter lumen 120 such that fluid flows over it through lumen 120 between the body cavity 92 and tube lumen 156. In some examples, flexible tensioning member 140 can pass through separate lumen(s), such as a separate lumen formed in catheter wall 122, or a separate lumen of an indwelling catheter 102 provided as a multi-lumen catheter.

It is to be understood that catheter assembly 100^{l} can be combined with examples of any of the catheter assemblies 100^{a}, 100^{b}, 100^{c}, 100^{d}, 100^{e}, 100^{f}, 100^{g}, 100^{h}, 100ⁱ, 100^{j} and/or 100^{k}, described throughout FIGS. 2A-12B hereinabove.

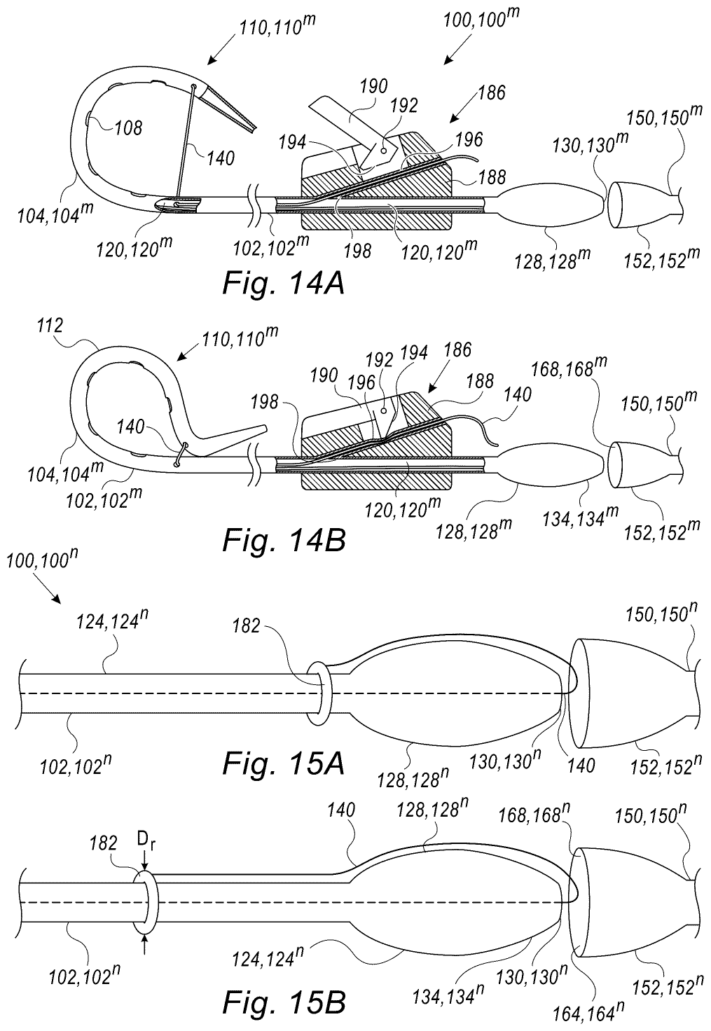

FIGS. 14A-14B show steps in utilization of a levered locker 186 of catheter assembly 100^{m}, which is an exemplary implementation of catheter assembly 100, and thus includes all of the features described for catheter assembly 100 that includes a pigtail 112. More specifically, catheter assembly 100^{m} can be similar to any example described above for catheter assembly 100^{l} with respect to FIGS. 13A-13D, except that instead of tying the flexible tensioning member 140 distal to catheter proximal expanded portion 128^{l}, the indwelling catheter 102^{m} further comprises a levered locker 186 for securing the flexible tensioning member 140 in a tensioned state.

Indwelling catheter 102^{m} comprises a levered locker 186 positioned about and connected to a portion of catheter 102^{m} which is distal to catheter proximal expanded portion 128^{m}, for securing the position of flexible tensioning member 140 with respect to the distal end portion 104^{m}, thereby maintaining the desired curled configuration of pigtail 112. Levered locker 186 includes a resilient sleeve 196 extending from sleeve passage 198 through locker body 188. The resilient sleeve 196 is in fluid communication, via sleeve passage 198, with the lumen the flexible tensioning member 140 extends through, such as catheter lumen 120, such that the free end of flexible tensioning member 140 extends from the lumen of catheter 102^{m}, through resilient sleeve 196, and out of levered locker 186.

The levered locker 186 further comprises a lever 190 pivotable connected to locker body 188 by pivot pin 192. Lever 190 can include an end that can be pressed (or released) by a user, and an inner cam surface 194 configured to contact and press against resilient sleeve 196.

FIG. 14A shows the levered locker 186 in a released state. In order to assume a curled configuration of the pigtail 112, the user can pull the free end of flexible tensioning member 140 extending out of the levered locker 186, and then pivotably push the lever 190, for example toward locker body 188, such that the inner cam surface 194 is press-locked against the resilient sleeve 196, squeezing over and locking the flexible tensioning member 140 in the desired tensioned configuration, as shown in FIG. 14B.

It is to be understood that catheter assembly 100^{m} can be combined with examples of any of the catheter assemblies 100^{a}, 100^{b}, 100^{c}, 100^{d}, 100^{e}, 100^{f}, 100^{g}, 100^{h}, 100ⁱ, 100^{j} and/or 100^{k}, described throughout FIGS. 2A-12B hereinabove.

FIGS. 15A-15B show steps in utilization of a frictionally movable ring 182 of catheter assembly 100ⁿ, which is an exemplary implementation of catheter assembly 100, and thus includes all of the features described for catheter assembly 100 that includes a pigtail 112. More specifically, catheter assembly 100ⁿ can be similar to any example described above for catheter assembly 100^{l} with respect to FIGS. 13A-13D, except that instead of tying the flexible tensioning member 140 distal to catheter proximal expanded portion 128^{l}, the indwelling catheter 102ⁿ further comprises a frictionally movable ring 182 for securing the flexible tensioning member 140 in a tensioned state.

In the illustrated example, the portion of flexible tensioning member 140 extending out of catheter proximal end portion 128ⁿ is attached to a frictionally movable ring 182 disposed over a portion of catheter 102ⁿ which is distal to catheter proximal expanded portion 128ⁿ. The movable ring 182 can be in the form of an O-ring, and is configured to be longitudinally movable along the outer surface of a portion of catheter 102^{m} distal to catheter proximal expanded portion 128^{m} when manually moved by a user, but to remain immovable in position when no external force is further applied thereto by the user, so as to retain the flexible tensioning member 140 in a desired position.

The flexible tensioning member 140 can exit from catheter proximal end opening 130ⁿ bend over catheter proximal edge 132ⁿ and extend distally over the catheter proximal end portion 128ⁿ, to connect with frictionally movable ring 182 (e.g., by gluing, tying, and the like). The user can manually push the ring 182 distally over catheter 102ⁿ, such as shown in the transition from FIG. 15A to FIG. 15B, so as to facilitate transition of the pigtail 112 to a curved configuration, similar to that shown in FIG. 13C for example. As the user releases the ring 182, the frictional force with the outer surface of catheter 102ⁿ is configured to prevent spontaneous displacement thereof. The external tube 150ⁿ can then be slid over or into catheter 102ⁿ.

It is to be understood that a catheter assembly 100ⁿ which includes a frictionally movable ring 182 can be combined with examples of any of the catheter assemblies 100^{a}, 100^{b}, 100^{c}, 100^{d}, 100^{e}, 100^{f}, 100^{j} and/or 100^{k}, described throughout FIGS. 2A-12B hereinabove.

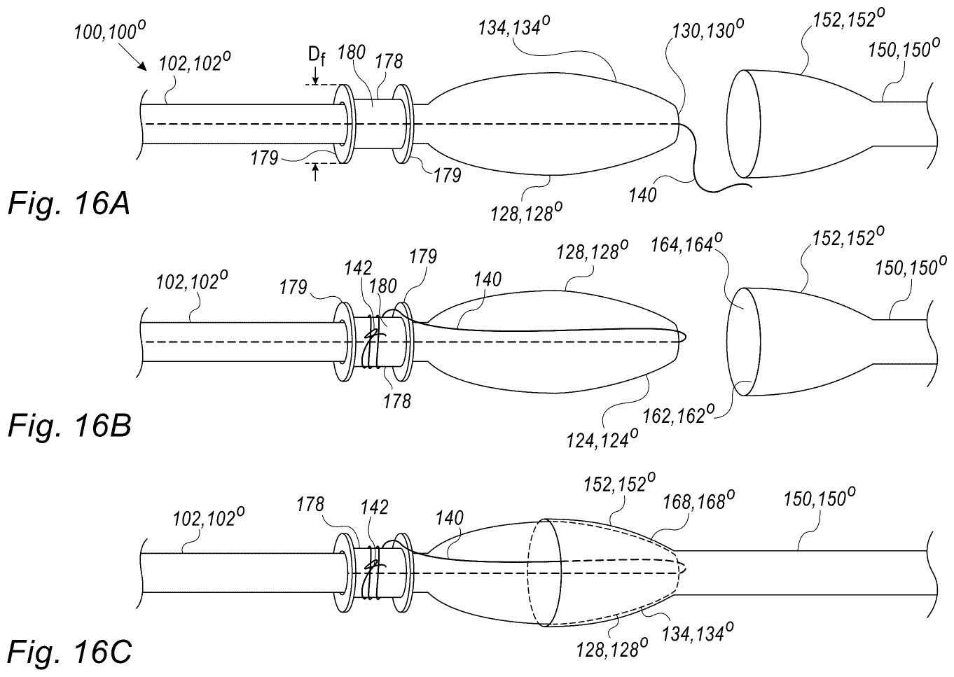

FIGS. 16A-16C show steps in utilization of a spool 178 of catheter assembly 100^{o}, which is an exemplary implementation of catheter assembly 100, and thus includes all of the features described for catheter assembly 100 that includes a pigtail 112. More specifically, catheter assembly 100^{o} can be similar to any example described above for catheter assembly 100^{l} with respect to FIGS. 13A-13D, except that instead of tying the flexible tensioning member 140 distal to catheter proximal expanded portion 128^{l}, the indwelling catheter 102^{o} further comprises spool 178 over which the flexible tensioning member 140 can be wrapped.

In the illustrated example, the indwelling catheter 102^{o} further comprises a spool 178 attached to a portion of catheter 102^{o} which is distal to catheter proximal expanded portion 128^{o}. The spool 178 can be affixed or otherwise attached to (e.g., glued or press-fit over) the outer surface of a portion of catheter 102^{o} distal to catheter proximal expanded portion 128^{o}. The spool can include two flanges 179 and a recessed core 180 disposed therebetween, over which the free end of the flexible tensioning member 140, optionally exiting from catheter proximal end opening 130^{o} (see FIG. 16A) and bent over catheter proximal edge 132^{o} to extend distally over the catheter proximal end portion 128^{o} (see FIG. 16B), can be wrapped and tied to, such that a knot 142 resides between flanges 179, as shown in FIG. 16B. The external tube 150^{o} can then be slid over or into catheter 102^{o}, as shown in FIG. 16C.

The flanges 179 can define a flange diameter D_{f}, which can be greater than the tube outer diameter Dₜₒ, in some examples, so as to prevent the tube 150^{o} from sliding over the spool 178 it its tube distal expanded portion 152 extends past the catheter proximal expanded portion 128.

In some implementations, the spool 178 includes only two flanges 179 longitudinally spaced from each other, without a recessed core 180 extending therebetween, such that the flexible tension member 140 can be wrapped around a portion of the outer surface of catheter 102^{o} bound between both flanges 179, with the knot 142 similarly formed between the flanges 179.

It is to be understood that a catheter assembly 100^{o} which includes a spool 178, can be combined with examples of any of the catheter assemblies 100^{a}, 100^{b}, 100^{c}, 100^{d}, 100^{e}, 100^{f}, 100^{g}, 100^{h}, 100ⁱ, 100^{j} and/or 100^{k}, described throughout FIGS. 2A-12B hereinabove.

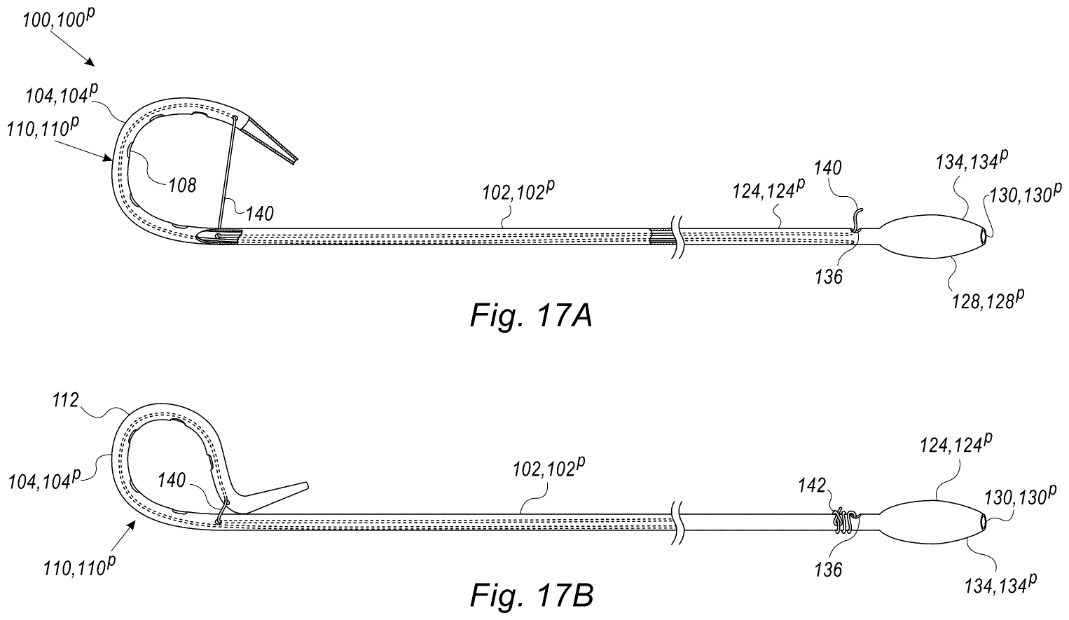

FIGS. 17A-17B show stages of utilizing an anchoring mechanism 110^{p} of catheter assembly 100^{p}, which is an exemplary implementation of disengageable catheter assembly 100, and thus includes all of the features described for disengageable catheter assembly 100 throughout the current disclosure. More specifically, catheter assembly 100^{p} can be similar to any example described above for catheter assembly 100^{l} with respect to FIGS. 13A-13D, except that instead of extending through catheter proximal end opening 130^{l}, the flexible tension member 140 of indwelling catheter 102^{p} extends through a catheter proximal side opening 136.

FIGS. 17A and 17B shows stages which are equivalent to the stages shown in FIGS. 13B and 13C for indwelling catheter 102^{l}. Indwelling catheter 102^{p} includes a catheter proximal side opening 136 extending through the thickness of catheter wall 122^{p}, at a position which is distal to catheter proximal expanded portion 128^{p}. This allows the free end of flexible tension member 140 to exit from the lumen of catheter 102^{p} at a position which is distal to catheter proximal edge 132^{p}, without the need for the tension member 140 to extend all the way up to catheter proximal end opening 130^{p} and form a U-turn around catheter proximal edge 132^{p}. The position of catheter proximal side opening 136 can advantageously distance the flexible tension member father away from the tube distal expanded portion 152 in the engaged state. The catheter proximal side opening 136 is designed to seal against the tension member 140 extending therethrough, to prevent leakage through the side opening 136. In some examples, an insulating component (not shown), such as an insulating ring or cover, can be disposed around the catheter proximal side opening 136, such as when the tension member 140 is passed therethrough, to seal the opening around the tension member 140.

It is to be understood that catheter assembly 100^{o} provided with catheter proximal side opening 136 through which the flexible tension member 140 extends, can be combined with examples of any of the catheter assemblies 100^{a}, 100^{b}, 100^{c}, 100^{d}, 100^{e}, 100^{f}, 100^{g}, 100^{h}, 100ⁱ, 100^{j} 100^{k}, 100^{m}, 100ⁿ, and/or 100^{o}, described throughout FIGS. 2A-12B and 14A-16C hereinabove.

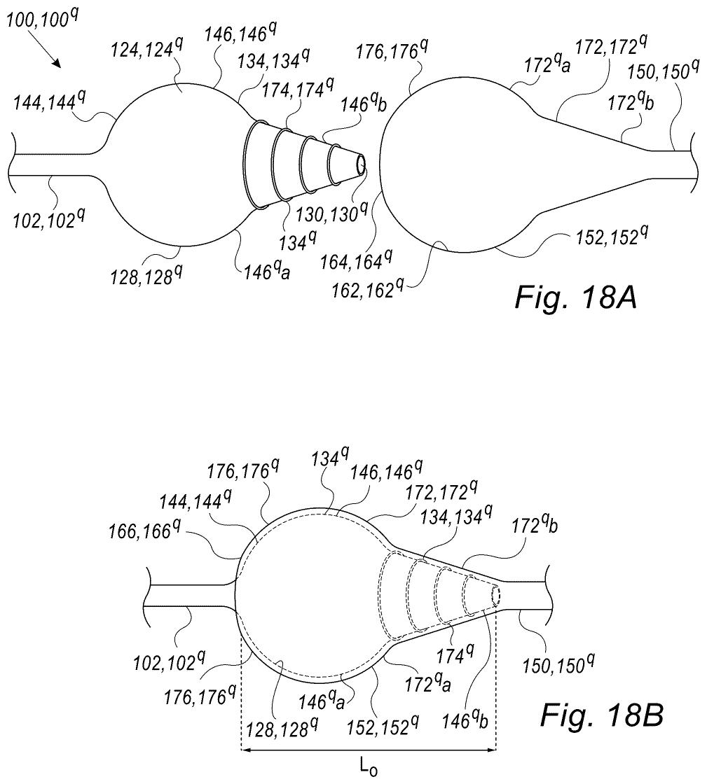

FIGS. 18A and 18B are side views in perspective of a portion of an exemplary disengageable catheter assembly 100^{q} shown in a disengaged state and an engaged state, respectively. Catheter assembly 100^{q} is an exemplary implementation of catheter assembly 100, and thus includes all of the features described for catheter assembly 100 and any components thereof throughout the current disclosure, except that the varying-diameter engagement surfaces 134^{q} and 168^{q} are formed over surfaces of multi-segmented expanded portions 128^{q} and 152^{q}, each optionally provided with differently-shaped segments.

In the illustrated example, the catheter proximal expanded portion 128^{q} is formed to include a spherical segment extending proximally from the tubular segment of catheter 102^{q}, and a frustoconical segment extending proximally from the spherical segment. The spherical segment of catheter proximal expanded portion 128^{q} defines the catheter outwardly expanding segment 144^{q} and a first catheter inwardly tapering segment 146^{q}a, and the frustoconical segment defines a second catheter inwardly tapering segment 146^{a}b extending from the first catheter inwardly tapering segment 146^{q}a to the catheter proximal edge 132^{q}. A series of circumferential protrusions 174^{q} are also shown to be disposed around the frustoconically-shaped second catheter inwardly tapering segment 146^{a}b.

The tube distal expanded portion 152^{q} has a complementary overall shape, formed to include a spherical segment extending proximally from the tube distal edge 166^{q}, and a frustoconical segment extending proximally from the spherical segment. The spherical segment of tube distal expanded portion 152^{q} defines the tube inwardly tapering segment 176^{q} and a first tube outwardly expanding segment 172^{q}a, and the frustoconical segment defines a second tube outwardly expanding segment 172^{a}b extending from the first tube outwardly expanding segment 172^{q}a to the tubular portion of external tube 150^{q}.

As shown in FIG. 18B, this configuration resembles a ball-and-socket joint coupling in the engaged state, wherein the cylindrical segment of the catheter proximal expanded portion 128^{q} can be the equivalent of a ball and the cylindrical segment of the tube distal expanded portion 152^{q} can be the equivalent of a socket, providing increased flexibility by which the tube 150^{q} can pivotably move in various direction, relative to catheter 102^{q}. Moreover, the ball-and-socket coupling can increase engagement between the tube 150^{q} and catheter 102^{q}, such that relatively smaller-dimensioned catheter proximal expanded portion 128^{q} and tube distal expanded portion 152^{q} can be used to achieve similar engagement that maintains both components coupled to each other at a force lower than the maximal non-separating pull force, and allowing disengagement at a force exceeding the threshold separating force. In some examples, a tubular section of the catheter 102^{q}, extending proximally from the catheter proximal expanded portion 128^{q}, is elastic enough to allow the transition region at which this tubular section is joined to the expanded portion 128^{q} to bend or flex in any desired direction, thus adding additional degrees of freedom to the movement of the catheter proximal expanded portion 128^{q} relative to a central axis extending through the tubular portion of the catheter 102^{q}.

It is to be understood that spherical segments are shown in combination with frustoconical segment by way of illustration and not limitation, and that any of the catheter proximal expanded portion 128 and/or tube distal expanded portion 152 can be provided with a single spherical section, or a spherical section combined with one or more other types of sections. Similarly, it is to be understood that the addition of circumferential protrusions 174^{q} to the frustoconical segment is shown by way of example, and that any of the catheter proximal expanded portion 128 and tube distal expanded portion 152 can be provided without circumferential protrusions 174^{q}, or with otherwise-shaped protrusions 174.

It is to be understood that a catheter assembly 100 formed to include a ball-and-socket type of connection, can be combined with examples of any of the catheter assemblies 100^{a}, 100^{b}, 100^{c}, 100^{d}, 100^{e}, 100^{f}, 100^{g}, 100^{h}, 100ⁱ, 100^{j} 100^{k}, 100^{l}, 100^{m}, 100ⁿ, 100^{o} and/or 100^{p}, described throughout FIGS. 2A-17B hereinabove.

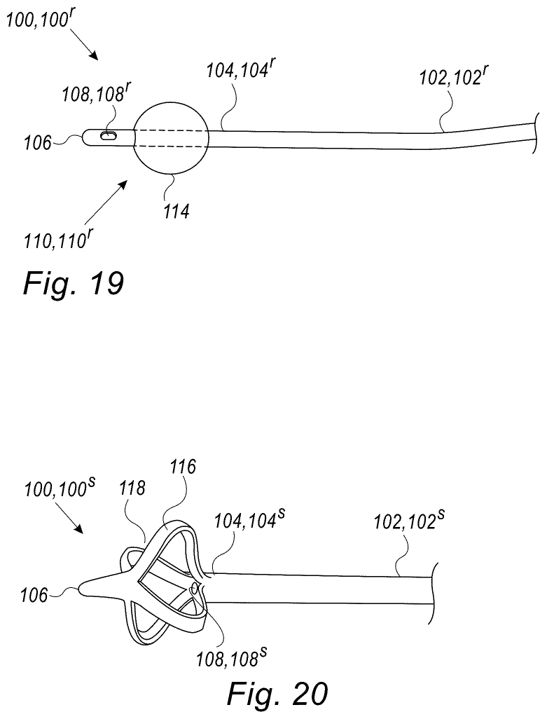

Catheter distal portion 104 can include any anchoring mechanism 110 configured to transition it between a compacted configuration and an expanded configuration, as described above. FIG. 19 shows a partial view of a distal section of a disengageable catheter assembly 100^{r} that includes an indwelling catheter which is implemented as a Foley catheter 102^{r}, provided with an anchoring mechanism 110^{r} that comprises an inflatable balloon 114. In such implementations, an inflatable balloon 114 is attached to catheter distal portion 104^{r}. The catheter 102^{r} is advanced into the body cavity 92 in a compacted or deflated state of the balloon 114, and the balloon 114 is then inflated by injection of suitable fluid (e.g., sterile water or saline) to a fully expanded or inflated configuration thereof. When inflated, the balloon rests on the inner wall of organ 90 and is significantly larger than the entry hole into organ 90. Consequently, indwelling catheter 102^{r} maintains it position within body cavity 92.

In some application of catheter assemblies 100, the maximal size of inflatable balloon 114 is limited by the small volume available by the body cavity 92. For example, the available volume of the inner cavity of the kidney 90, for catheter assemblies 100 implemented as nephrostomy catheters, can be significantly less than the volume available by other organs for other applications, such as the volume in the stomach for implementing catheter assemblies 100 as feed catheters. The limited maximal size of balloons 114 in some such implementations can increase the risk of balloon dislodgment from the organ 90, even in a fully inflated configuration thereof.

It is to be understood that catheter assembly 100^{r} provided with a Foley catheter 102^{r} can be combined with examples of any of the catheter assemblies (excluding those implemented to include pigtail catheters) 100^{a}, 100^{b}, 100^{c}, 100^{d}, 100^{e}, 100^{f}, 100^{j}, 100^{k} and/or 100^{q}, described throughout FIGS. 2A-12B and 18A-18B hereinabove.

FIG. 20 shows a partial view of a distal section of a disengageable catheter assembly 100^{s} that includes an indwelling catheter which is implemented as a Malecot catheter 102^{s}, provided with an anchoring mechanism 110^{s} that comprises Malecot ribs 116. In such implementations, the catheter distal portion 104^{s} can include longitudinal slits that form inter-rib spacings 118 between Malecot ribs 116. Malecot ribs 116 can be pre-shaped to assume an expanded shape, as shown for example in FIG. 20, wherein a guidewire or other rod member can be utilized to push the catheter distal tip 106 to maintain the ribs 116 in a relatively straight compacted configuration during delivery, and then retrieved to allow the ribs 116 to assume their expanded natural configuration. It is to be understood that catheter assembly 100^{s} provided with a Malecot catheter 102^{s} can be combined with examples of any of the catheter assemblies 100^{a}, 100^{b}, 100^{c}, 100^{d}, 100^{e}, 100^{f}, 100^{j}, 100^{k} and/or 100^{q}, described throughout FIGS. 2A-12B and 18A-18B hereinabove.

In some implementations, transitioning Malecot ribs 116 to the expanded configuration can be facilitated by pulling a proximal end of a flexible tension member 140, which can be attached to a portion of catheter distal portion 104^{s} (e.g., catheter distal tip 106) which is distal to the ribs 116, while the proximal end of Malecot ribs 116 remains stationary or unmoved relative to the flexible tension member 140, and the flexible tension member 140 can then be maintained in a tightened configuration, in a similar manner described above to pigtail catheters 102 that include flexible tension members 140. In such implementations, a catheter assembly 100^{s} provided with a Malecot catheter 1028 can be combined with examples of any of the catheter assemblies 100^{l}, 100^{m}, 100ⁿ, 100^{o} and/or 100^{p}, described throughout FIGS. 13A-17C hereinabove.

While several examples of anchoring mechanisms 110 are illustrated and described above, such as pigtail 112, inflatable balloon 114, and Malecot ribs 116, it is to be understood that any other type of an anchoring mechanism configured to transition between compacted and expanded configurations is contemplated, including, but not limited to, expandable stents, extendable arms, and the like.

It is to be understood that a catheter proximal expanded portion 128 of any exemplary disengageable catheter assembly 100 disclosed herein, can be either integrally formed with a tubular portion of the corresponding indwelling catheter 102, or provided as a separate component affixed to the proximal end of the tubular portion, such as by adhering, welding, press-fitting, and the like. A tubular portion of the corresponding indwelling catheter 102 is the portion of the catheter 102 extending distally from the expanded portion 128, which can be a portion of the catheter 102 having a uniform inner and/or outer diameter.

Similarly, it is to be understood that a tube distal expanded portion 152 of any exemplary disengageable catheter assembly 100 disclosed herein, can be either integrally formed with a tubular portion of the corresponding external tube 150, or provided as a separate component affixed to the distal end of the tubular portion, such as by adhering, welding, press-fitting, and the like. A tubular portion of the corresponding external tube 150 is the portion of the tube 150 extending proximally from the expanded portion 152, which can be a portion of the tube 150 having a uniform inner and/or outer diameter.

Thus, while in some examples, any exemplary indwelling catheter 102 and/or external tube 150 can be formed as unitary components that include a catheter proximal expanded portion 128 and/or a tube distal expanded portion 152 integrally extending from the respective tubular portions of the corresponding catheter 102 and/or tube 150, respectively, in some implementations, existing indwelling catheters 102 and/or external tube 150, formed as uniform tubular members, can be modified to serve as part of any of the exemplary disengageable catheter assemblies 100 described above by attaching separately formed components defining catheter proximal expanded portions 128 and/or a tube distal expanded portions 152 to the appropriate and of the existing tubes.

### Some Examples of the Disclosed Implementations

Some examples of above-described implementations are enumerated below. It should be noted that one feature of an example in isolation or more than one feature of the example taken in combination and, optionally, in combination with one or more features of one or more examples below are examples also falling within the disclosure of this application.

Example 1. A disengageable catheter assembly comprising:

an indwelling catheter configured to be inserted into a patient's body, the indwelling catheter comprising a catheter wall defining a catheter lumen, wherein the indwelling catheter extends between a catheter distal portion and a catheter proximal expanded portion, wherein the catheter distal portion comprises an anchoring mechanism configured to transition between a compacted configuration and an expanded configuration, wherein the catheter distal portion comprises at least one catheter distal opening in fluid communication with the catheter lumen, and wherein the catheter proximal expanded portion comprises a catheter varying-diameter engagement surface; and

an external tube comprising a tube wall defining a tube lumen, wherein the external tube comprises a tube distal expanded portion which comprises a tube varying-diameter engagement surface;

wherein the disengageable catheter assembly is configured to non-adjustably transition between an engaged state, in which the catheter engagement surface and the tube engagement surface are frictionally engaged with each other, and a disengaged state, in which the indwelling catheter and the external tube are separated from each other such that no portion of the external tube is disposed around or through the indwelling catheter;

wherein the catheter lumen is in fluid communication with the tube lumen in the engaged state;

wherein the frictional engagement between the catheter varying-diameter engagement surface and the tube varying-diameter engagement surface in the engaged state is configured to allow disengagement therebetween when the indwelling catheter and the external tube are subjected to an axial pull force exceeding a threshold separating force that is not greater than 20 N for a duration of no more than 10 seconds;

wherein, when the indwelling catheter is inserted into the patient's body, the catheter distal portion is configured to reside inside the patient's body, while the catheter proximal expanded portion and the entirety of the external tube are configured to remain external to the patient's body;

wherein engagement between the catheter varying-diameter engagement surface and the tube varying-diameter engagement surface in the engaged state is configured to be facilitated by a distally-oriented movement of the tube distal expanded portion towards the catheter proximal expanded portion; and

wherein the disengageable catheter assembly is manually reconnectable from the disengaged state to the engaged state by distally moving the tube distal expanded portion towards the catheter proximal expanded portion.

Example 2. The disengageable catheter assembly of claim 1, wherein the catheter proximal expanded portion comprises a catheter outwardly expanding segment, and a catheter inwardly tapering segment extending proximally from the catheter outwardly expanding segment, and wherein the tube distal expanded portion comprises a tube outwardly expanding segment terminating at a tube distal edge.

Example 3. The disengageable catheter assembly of claim 2, wherein the catheter proximal expanded portion has a prolate spheroid shape, and wherein the tube distal expanded portion has a prolate hemispheroid shape.

Example 4. The disengageable catheter assembly of claim 2, wherein the catheter inwardly tapering segment has a frustoconical shape, and wherein the tube outwardly expanding segment has a frustoconical shape.

Example 5. The disengageable catheter assembly of any one of claims 2-4, wherein the catheter inwardly tapering segment comprises a first catheter inwardly tapering segment and a second catheter inwardly tapering segment, and wherein the tube outwardly expanding segment comprises a first tube outwardly expanding segment and a second tube outwardly expanding segment.

Example 6. The disengageable catheter assembly of claim 2, wherein the tube distal expanded segment further comprises a tube inwardly tapering segment proximal to the tube outwardly expanding segment.

Example 7. The disengageable catheter assembly of claim 6, wherein the catheter proximal expanded portion comprises a spherical segment and wherein the tube distal expanded segment comprises a spherical segment.

Example 8. The disengageable catheter assembly of claim 1, wherein the catheter proximal expanded portion comprises a catheter outwardly expanding segment, and wherein the tube distal expanded portion comprises a tube outwardly expanding segment terminating at a tube distal edge, and a tube inwardly tapering segment proximal to the tube outwardly expanding segment.

Example 9. The disengageable catheter assembly of claim 8, wherein the tube distal expanded portion has a prolate spheroid shape, and wherein the catheter proximal expanded portion has a prolate hemispheroid shape.

Example 10. The disengageable catheter assembly of claim 8, wherein the catheter outwardly expanding segment has a frustoconical shape, and wherein the tube outwardly expanding segment has a frustoconical shape.

Example 11. The disengageable catheter assembly of any one of claims 1-10, wherein the catheter proximal expanded portion comprises at least one catheter flat.

Example 12. The disengageable catheter assembly of any one of claims 1-10, wherein the tube distal expanded portion comprises at least one tube flat.

Example 13. The disengageable catheter assembly of any one of claims 1-12, wherein the threshold separating force is not higher than 15 N.

Example 14. The disengageable catheter assembly of any one of claims 1-12, wherein the threshold separating force is not higher than 10 N.

Example 15. The disengageable catheter assembly of any one of claims 1-12, wherein the threshold separating force is not higher than 7 N.

Example 16. The disengageable catheter assembly of any one of claims 1 to 15, wherein the catheter varying-diameter engagement surface and the tube varying-diameter engagement surface are configured, when in the engaged state, to maintain frictional engagement therebetween, as long as an axial pull force applied to the external tube and the indwelling catheter does not exceed a maximal non-separating pull force, wherein the maximal non-separating pull force is less than the threshold separating pull force.

Example 17. The disengageable catheter assembly of claim 16, wherein the maximal non-separating pull force is equal to or less than 95% of the threshold separating pull force.

Example 18. The disengageable catheter assembly of claim 16, wherein the maximal non-separating pull force is equal to or less than 85% of the threshold separating pull force.

Example 19. The disengageable catheter assembly of claim 16, wherein the maximal non-separating pull force is equal to or less than 75% of the threshold separating pull force.

Example 20. The disengageable catheter assembly of any one of claims 1 to 19, wherein the disengageable catheter assembly is devoid of a connector configured to facilitate connection of the indwelling catheter and the external tube to each other in any type of engagement which is not frictional engagement.

Example 21. The disengageable catheter assembly of any one of claims 1 to 20, wherein the disengageable catheter assembly is devoid of a frangible connector configured to break in response to a pull force equal to or greater than the threshold separating force.

Example 22. The disengageable catheter assembly of any one of claims 1 to 21, wherein an inner diameter of the tube distal expanded portion is less than an outer diameter of the catheter proximal expanded portion at any axial position of contact between the tube varying-diameter engagement surface and the catheter varying-diameter engagement surface in the engaged state.

Example 23. The disengageable catheter assembly of any one of claims 1 to 21, wherein an inner diameter of the catheter proximal expanded portion is less than an outer diameter of the tube distal expanded portion at any axial position of contact between the catheter varying-diameter engagement surface and the tube varying-diameter engagement surface in the engaged state.

Example 24. The disengageable catheter assembly of any one of claims 1 to 23, wherein the catheter varying-diameter engagement surface is rougher than at least one other surface of the indwelling catheter.

Example 25. The disengageable catheter assembly of any one of claims 1 to 24, wherein the tube varying-diameter engagement surface is rougher than at least one other surface of the external tube.

Example 26. The disengageable catheter assembly of any one of claims 1 to 25, wherein the indwelling catheter further comprises at least one friction layer attached to the catheter proximal expanded portion, wherein the friction layer comprises the catheter varying-diameter engagement surface.

Example 27. The disengageable catheter assembly of any one of claims 1 to 25, wherein the external tube further comprises at least one friction layer attached to the tube distal expanded portion, wherein the friction layer comprises the tube engagement surface.

Example 28. The disengageable catheter assembly of any one of claims 1 to 27, wherein the catheter proximal expanded portion comprises one or more protrusions extending radially from a corresponding surface of the catheter proximal expanded portion, wherein the one or more protrusions define the catheter varying-diameter engagement surface.

Example 29. The disengageable catheter assembly of any one of claims 1 to 27, wherein the tube distal expanded portion comprises one or more protrusions extending radially from a corresponding surface of the tube distal expanded portion, wherein the one or more protrusions define the tube varying-diameter engagement surface.

Example 30. The disengageable catheter assembly of claim 28 or 29, wherein the one or more protrusions comprises a plurality of circumferential protrusions axially spaced from each other.

Example 31. The disengageable catheter assembly of claim 28 or 29, wherein the one or more protrusions comprises a plurality of axial protrusions circumferentially spaced from each other.

Example 32. The disengageable catheter assembly of any one of claims 1 to 31, wherein the anchoring mechanism comprises a pigtail.

Example 33. The disengageable catheter assembly of claim 32, wherein the catheter distal portion is pre-shaped to assume a curved configuration in a free state thereof.

Example 34. The disengageable catheter assembly of any one of claims 1 to 31, wherein the anchoring mechanism comprises Malecot ribs.

Example 35. The disengageable catheter assembly of claim 34, wherein the Malecot ribs are pre-shaped to assume an expanded configuration in a free state thereof.

Example 36. The disengageable catheter assembly of claim 32 or 35, wherein the indwelling catheter further comprises a flexible tensioning member attached to the catheter distal portion and configured to transition the anchoring mechanism to the expanded configuration when a portion of the flexible tensioning member, extending away from the catheter wall at or proximate to the catheter proximal expanded portion, is pulled relative to the catheter distal portion.

Example 37. The disengageable catheter assembly of claim 36, wherein a free ended portion of the flexible tensioning member extends out of a catheter proximal end opening of the indwelling catheter.

Example 38. The disengageable catheter assembly of claim 36, wherein a free ended portion of the flexible tensioning member extends out of a catheter proximal side opening of the indwelling catheter.

Example 39. The disengageable catheter assembly of any one of claims 36 to 38, further comprising a levered locker positioned distal to the catheter proximal expanded portion, the levered locker comprising:

a resilient sleeve extending through a locker body of the lever locker, the resilient sleeve configured to allow the flexible tensioning member to extend from the indwelling catheter therethrough; and

a lever pivotably attached to the lever body, the lever comprising an inner cam surface configured to squeeze the resilient sleeve and lock the flexible tensioning member passing therethrough.

Example 40. The disengageable catheter assembly of any one of claims 36 to 38, further comprising a frictionally movable ring to which a proximal end of the flexible tensioning member is attached, wherein the frictionally movable ring is configured to be longitudinally movable along a portion of the indwelling catheter distal to the catheter proximal expanded portion, in response to manual force applied thereto, but remains immovable in position when no manual force is applied thereto.

Example 41. The disengageable catheter assembly of any one of claims 36 to 38, further comprising a spool comprising a pair of flanges attached to a portion of the indwelling catheter distal to the catheter proximal expanded portion, wherein a knot of the flexible tension member is situated between the flanges in the engaged state.

Example 42. The disengageable catheter assembly of any one of claims 1 to 31, wherein the anchoring mechanism comprises an inflatable balloon.

Example 43. The disengageable catheter assembly of any one of claims 1 to 42, wherein the frictional engagement between the catheter varying-diameter engagement surface and the tube varying-diameter engagement surface in the engaged state is configured to allow disengagement therebetween when the indwelling catheter and the external tube are subjected to an axial pull force exceeding a threshold separating force that is not greater than 20 N for a duration of no more than 5 seconds.

It is appreciated that certain features of the invention, which are, for clarity, described in the context of separate examples, may also be provided in combination in a single example. Conversely, various features of the invention, which are, for brevity, described in the context of a single example, may also be provided separately or in any suitable sub-combination or as suitable in any other described example of the invention. No feature described in the context of an example is to be considered an essential feature of that example, unless explicitly specified as such.

In view of the many possible examples to which the principles of the disclosure may be applied, it should be recognized that the illustrated examples are only preferred examples and should not be taken as limiting the scope. Rather, the scope is defined by the following claims. We therefore claim all that comes within the scope and spirit of these claims.

1. A disengageable catheter assembly comprising:
an indwelling catheter configured to be inserted into a patient's body, the indwelling catheter comprising a catheter wall defining a catheter lumen, wherein the indwelling catheter extends between a catheter distal portion and a catheter proximal expanded portion, wherein the catheter distal portion comprises an anchoring mechanism configured to transition between a compacted configuration and an expanded configuration, wherein the catheter distal portion comprises at least one catheter distal opening in fluid communication with the catheter lumen, and wherein the catheter proximal expanded portion comprises a catheter varying-diameter engagement surface; and
an external tube comprising a tube wall defining a tube lumen, wherein the external tube comprises a tube distal expanded portion which comprises a tube varying-diameter engagement surface;
wherein the disengageable catheter assembly is configured to non-adjustably transition between an engaged state, in which the catheter engagement surface and the tube engagement surface are frictionally engaged with each other, and a disengaged state, in which the indwelling catheter and the external tube are separated from each other such that no portion of the external tube is disposed around or through the indwelling catheter;
wherein the catheter lumen is in fluid communication with the tube lumen in the engaged state;
wherein the frictional engagement between the catheter varying-diameter engagement surface and the tube varying-diameter engagement surface in the engaged state is configured to allow disengagement therebetween when the indwelling catheter and the external tube are subjected to an axial pull force exceeding a threshold separating force that is not greater than 20 N for a duration of no more than 10 seconds;
wherein, when the indwelling catheter is inserted into the patient's body, the catheter distal portion is configured to reside inside the patient's body, while the catheter proximal expanded portion and the entirety of the external tube are configured to remain external to the patient's body;
wherein engagement between the catheter varying-diameter engagement surface and the tube varying-diameter engagement surface in the engaged state is configured to be facilitated by a distally-oriented movement of the tube distal expanded portion towards the catheter proximal expanded portion; and
wherein the disengageable catheter assembly is manually reconnectable from the disengaged state to the engaged state by distally moving the tube distal expanded portion towards the catheter proximal expanded portion; and
wherein the disengageable catheter assembly is devoid of a connector configured to facilitate connection of the indwelling catheter and the external tube to each other in any type of engagement which is not frictional engagement.

2. The disengageable catheter assembly of claim 1, wherein the catheter proximal expanded portion comprises a catheter outwardly expanding segment, and a catheter inwardly tapering segment extending proximally from the catheter outwardly expanding segment, and wherein the tube distal expanded portion comprises a tube outwardly expanding segment terminating at a tube distal edge.

3. The disengageable catheter assembly of claim 2, wherein the catheter proximal expanded portion has a prolate spheroid shape, and wherein the tube distal expanded portion has a prolate hemispheroid shape.

4. The disengageable catheter assembly of claim 2, wherein the catheter inwardly tapering segment has a frustoconical shape, and wherein the tube outwardly expanding segment has a frustoconical shape.

5. The disengageable catheter assembly of claim 2, wherein the catheter inwardly tapering segment comprises a first catheter inwardly tapering segment and a second catheter inwardly tapering segment, and wherein the tube outwardly expanding segment comprises a first tube outwardly expanding segment and a second tube outwardly expanding segment.

6. The disengageable catheter assembly of claim 2, wherein the tube distal expanded segment further comprises a tube inwardly tapering segment proximal to the tube outwardly expanding segment.

7. The disengageable catheter assembly of claim 6, wherein the catheter proximal expanded portion comprises a spherical segment and wherein the tube distal expanded segment comprises a spherical segment.

8. The disengageable catheter assembly of claim 1, wherein the catheter proximal expanded portion comprises a catheter outwardly expanding segment, and wherein the tube distal expanded portion comprises a tube outwardly expanding segment terminating at a tube distal edge, and a tube inwardly tapering segment proximal to the tube outwardly expanding segment.

9. The disengageable catheter assembly of claim 8, wherein the tube distal expanded portion has a prolate spheroid shape, and wherein the catheter proximal expanded portion has a prolate hemispheroid shape.

10. The disengageable catheter assembly of claim 8, wherein the catheter outwardly expanding segment has a frustoconical shape, and wherein the tube outwardly expanding segment has a frustoconical shape.

11. The disengageable catheter assembly of claim 1, wherein the threshold separating force is not higher than 15 N.

12. The disengageable catheter assembly of claim 1, wherein the catheter varying-diameter engagement surface and the tube varying-diameter engagement surface are configured, when in the engaged state, to maintain frictional engagement therebetween, as long as an axial pull force applied to the external tube and the indwelling catheter does not exceed a maximal non-separating pull force, wherein the maximal non-separating pull force is less than the threshold separating pull force.

13. The disengageable catheter assembly of claim 12, wherein the maximal non-separating pull force is equal to or less than 75% of the threshold separating pull force.

14. The disengageable catheter assembly of claim 1, wherein the disengageable catheter assembly is devoid of a frangible connector configured to break in response to a pull force equal to or greater than the threshold separating force.

15. The disengageable catheter assembly of claim 1, wherein the indwelling catheter further comprises at least one friction layer attached to the catheter proximal expanded portion, wherein the friction layer comprises the catheter varying-diameter engagement surface.

16. The disengageable catheter assembly of claim 1, wherein the external tube further comprises at least one friction layer attached to the tube distal expanded portion, wherein the friction layer comprises the tube engagement surface.

17. The disengageable catheter assembly of claim 1, wherein the catheter proximal expanded portion comprises one or more protrusions extending radially from a corresponding surface of the catheter proximal expanded portion, wherein the one or more protrusions define the catheter varying-diameter engagement surface.

18. The disengageable catheter assembly of claim 1, wherein the tube distal expanded portion comprises one or more protrusions extending radially from a corresponding surface of the tube distal expanded portion, wherein the one or more protrusions define the tube varying-diameter engagement surface.

19. The disengageable catheter assembly of claim 1, wherein the indwelling catheter further comprises a flexible tensioning member attached to the catheter distal portion and configured to transition the anchoring mechanism to the expanded configuration when a portion of the flexible tensioning member, extending away from the catheter wall at or proximate to the catheter proximal expanded portion, is pulled relative to the catheter distal portion.

20. The disengageable catheter assembly of claim 1, wherein the frictional engagement between the catheter varying-diameter engagement surface and the tube varying-diameter engagement surface in the engaged state is configured to allow disengagement therebetween when the indwelling catheter and the external tube are subjected to an axial pull force exceeding a threshold separating force that is not greater than 20 N for a duration of no more than 5 seconds.
